(19) United States
(12) Patent Application Publication
(10) Pub. No.: US 2007/0154572 A1
(43) Pub. Date: Jul. 5, 2007

(54) Compositions derived from portulaca oleracea l. and methods of using same for modulating blood glucose levels

(76) Inventors: Sobhi Sauob, Doar-Na Galil Tachton (IL); Jan Van Mil, D. N. Menashe (IL)

Correspondence Address: Martin D Moynihan;Prtsi Inc, PO Box 16446, Arlington, VA, 22215, US

(21) Appl. No.: 10/583,759

(22) PCT Filed: Dec. 22, 2004
(86) PCT No.: PCT/IL04/01155

§ 371 Date: Jun. 21, 2006

Related U.S. Application Data
(60) Provisional application No. 60/530,918, filed on Dec. 22, 2003.

(51) Int. Cl. A61K 36/185

(52) U.S. Cl. 424/725

(57) ABSTRACT

Compositions including anti hyperglycemic agents from Portulaca oleracea L. are provided. Also provided are methods of isolating such anti hyperglycemic agents and methods of using same.

### FIELD AND BACKGROUND OF THE INVENTION

The present invention relates to compositions from Portulaca oleracea L. and, more particularly, to methods of using same for modulating blood glucose levels.

Diabetes mellitus is a serious chronic metabolic disorder that has a significant impact on the health, quality of life and life expectancy of patients as well as on the health care system. In the United States, diabetes is the sixth leading cause of death [National Institute of Diabetes and digestive and kidney diseases (1995) Diabetes Statistics. Bethesda, Md.: NIDDM NIH Publication no. 96-3926].

Diabetes is divided into two major categories: type I diabetes (formerly known as insulin-dependent diabetes mellitus or IDDM) and type II diabetes (formerly known as non-insulin dependent diabetes mellitus or NIDDM). The overall prevalence of diabetes is approximately 6% of the population, of which 90% is type II [Diabetes (1996) Vital Statistics. Alexandria, Va.:American Diabetes Association]. Treatment and care of diabetes represents a substantial portion of the national health care expenditure, over 105 $ billion annually.

Type II diabetes represents a syndrome with disordered metabolism of carbohydrate and fat. The most prominent clinical feature is hyper-glycemia manifested by fasting plasma glucose level above 126 mg/dl, or glycosylated hemoglobin A_{1c} (HbA_{1c}) greater than 6.9%. In most patients with type II diabetes, the onset is in adulthood, most commonly in obese people over 40 years of age. Hypertension, hyperlipidemia, hyperinsulinemia and atherosclerosis are often associated with diabetes.

The early stage of type II diabetes is characterized by insulin resistance in insulin-targeting tissues, mainly the liver, skeletal muscle and adipocytes. Insulin resistance in these tissues is associated with excessive glucose production by the liver and impaired glucose utilization by peripheral tissues, especially muscle. These events undermine metabolic homeostasis, but may not directly lead to overt diabetes in the early stage. With increased insulin secretion to compensate for insulin resistance, baseline blood glucose levels can be maintained within normal ranges, but the patients may demonstrate impaired responses to prandial carbohydrate loading and to oral glucose tolerance tests. The chronic overstimulation of insulin secretion gradually diminishes and eventually exhausts the islet β cell reserve. A state of absolute insulin deficiency ensues and leading to overt diabetes [DeFronzo (1988) Diabetes 37:667-687; Seely (1993) Moller D, ed. Insulin Resistance and its clinical Disorders. London England: John Wiley & Sons, Ltd; 187-252]. The rate of transition from impaired glucose tolerance to type II diabetes is highly influenced by the genetic background, obesity, distribution of body fat, sedentary lifestyle, aging and concomitant medical conditions [Clark (1998) Diabetes care 21:C32-C34].

The life quality of Type II diabetic patients with chronic and severe hypoglycemia is severely affected. Typical symptoms include tiredness and lethargy which can become severe leading to a decrease in work performance in adults and increase a falls in the elderly. Acute complications include metabolic problems and infection. The long-term complications are macrovascular complications (e.g., hypertension, dyslipidemia, myocardial infarction, stroke), microvascular complications (e.g., retinopathy, nephropathy, diabetic neuropathy, diarrhea, neurogenic bladder, impaired cardiovascular reflexes, sexual dysfunction) and foot disorders.

Conventional treatment of type II diabetes is focused at lifestyle management. In addition to exercise, weight control and medical nutrition therapy, oral glucose lowering drugs and injections of insulin are the conventional therapies. Pharmacological treatment is indicated when fasting glucose level exceeds 140 mg/dl, the postprandial glucose level exceeds 160 mg/dl or HbA_{1c} exceeds 8%.

Currently available oral agents for treating Type II diabetes include first and second generations Sulfonylureas, which enhance insulin secretion from pancreatic β cells; Biguanides (e.g., metformin®) originally derived from a medicinal plant Galega Officinalis, which reduces plasma glucose by inhibiting hepatic glucose production and increasing muscle glucose uptake; a glucosidase inhibitors (e.g., ascorbate) which decrease post-prandial glucose levels by interfering with carbohydrate digestion and delaying gastrointestinal absorption of glucose; Thiazolidinediones (e.g., troglitazone®, rosiglitazone® and pioglitazone®), which improve insulin sensitivity in muscle and in the liver; and Meglitinides (e.g., Repaglinide®) which augment insulin secretion.

Although initial responses to the above described oral hypoglycemic drugs have been satisfactory, these drugs lose their effectiveness in a significant percentage of treated patients and treatment is usually accompanied by adverse side effects such as weight gain, hypoglycemia, gastrointestinal disturbances, liver toxicity and high LDL cholesterol [Dey (2002) Alternative Medicine Review 7:45-58 and references therein]. For these reasons insulin is usually added to the oral agent when glycemic control is suboptimal at maximal doses of oral medications. However, weight gain and hypoglycemia are common side effects of insulin therapy

In light of the severe limitations which accompany conventional therapies, alternative approaches for treating diabetes, such as herbal medications with antihyperglycemic activities are increasingly sought by patients and health care professionals. To date over 400 traditional plant treatments for diabetes have been reported [Bailey (1989) Diabetes Care 12:553-564], though only a small number thereof have undergone scientific and medical evaluation in order to assess their efficacy. The most commonly used medicinal herbs for treating diabetes include the Ginseng species such as the Asian and American ginseng species which have been reported to have significant hypoglycemic action; Momordica charantia (Bitter melon), which is used widely in folk medicines as a remedy for diabetes; Trigonella foenum graecum (Fenugreek), which has been used as a remedy for diabetes, particularly in India; Gymnema sylvestre; Allium cepa (onions) and Allium sativum (garlic) which have blood sugar lowering effects derived from volatile oils which are present in the raw onion and garlic cloves; Pterocarpus marsupium and other epicatechin-containing plants; Aloe vera and others.

Portulaca oleracea L., also known as Purslane, Verdolaga and Pursley, is an edible succulent ‘weed’ which is cultivated in most parts of the world.

Portulaca oleracea L. contains many biologically active compounds as well as many nutrients, including alkaloids, omega-3 fatty acids, coumarins, flavonoids and anthraquinone glycosides (2).

The plant has been traditionally used as a remedy for a wide variety of ailments, in particular as a treatment against parasites, and digestive disorders. In addition, anti-inflammatory and anti-fungal activities have been associated with Portulaca oleracea L. Unverified reports from around the world demonstrated the use of purslane as a remedy for many ailments and conditions (3).

Portulaca oleracea L. has been previously reported as a remedy for hyperglycemia. Eskander and H. Won Jun (4) showed the efficacy of Portulaca oleracea L. (whole plant) in reducing glucose levels in blood. PCT Appl. No. 00/00211 teaches the use of hydrocolloids extracted from Portulaca oleracea L. for reducing sugar level in the blood.

While reducing the present invention to practice, the present inventors uncovered that polar and non-polar extracts of Portulaca oleracea L. can be efficiently used to modulate blood glucose levels in subjects in need thereof in a biosafe manner.

### SUMMARY OF THE INVENTION

According to one aspect of the present invention there is provided a method of isolating anti hyperglycemic agents from Portulaca oleracea L., the method comprising extracting polar components from Portulaca oleracea L., thereby isolating the anti hyperglycemic agents from Portulaca oleracea L.

According to further features in preferred embodiments of the invention described below, the extracting is effected by employing a solvent gradient of increasing polarity

According to still further features in the described preferred embodiments the extracting is effected by ethanol-water extraction.

According to still further features in the described preferred embodiments the solvents of increasing polarity are hexane, ethyl acetate, dichloromethane, methanol and water.

According to still further features in the described preferred embodiments the solvents of increasing polarity are hexane:dichloromethane:ethylacetate (1:1:1) and methanol:ethanol:water (1:1:1).

According to still further features in the described preferred embodiments the method further comprising purifying the polar components from the extract.

According to another aspect of the present invention there is provided a method of isolating anti hyperglycemic agents from Portulaca oleracea L., the method comprising extracting non-polar components from Portulaca oleracea L., thereby purifying the anti hyperglycemic agents from Portulaca oleracea L.

According to still further features in the described preferred embodiments extracting is effected using non-polar solvents.

According to still further features in the described preferred embodiments the extracting is effected by ethanol-water extraction.

According to still further features in the described preferred embodiments the non-polar solvents are selected from the group consisting of hexane, dichloromethane and ethyl acetate.

According to still further features in the described preferred embodiments the method further comprising purifying the non-polar components from the extract.

According to still further features in the described preferred embodiments the purifying the non-polar components from the extract is effected by thin layer chromatography.

According to yet another aspect of the present invention there is provided a composition of matter comprising an ethanol-water extract of Portulacea oleracea L.

According to still another aspect of the present invention there is provided a composition of matter comprising a polar fraction extract of Portulacea oleracea L.

According to an additional aspect of the present invention there is provided use of a composition including a polar extract of Portulaca oleracea L. for reducing blood glucose levels.

According to yet an additional aspect of the present invention there is provided a composition-of-matter comprising a non-polar fraction extract of Portulaca oleracea L.

According to still an additional aspect of the present invention there is provided use of a composition including a non-polar extract of Portulaca oleracea L. for decreasing blood glucose levels.

According to a further aspect of the present invention there is provided use of a composition including ethanol-water extract of Portulaca oleracea L. for decreasing blood glucose levels.

According to yet a further aspect of the present invention there is provided a pharmaceutical composition for reducing blood glucose levels comprising a therapeutic effective amount of a composition including a polar fraction extract of Portulaca oleracea L. and a pharmaceutical acceptable carrier or diluent.

According to still a further aspect of the present invention there is provided a pharmaceutical composition for decreasing blood glucose levels comprising a therapeutic effective amount of a composition including a non-polar fraction extract of Portulaca oleracea L. and a pharmaceutical acceptable carrier or diluent.

According to still a further aspect of the present invention there is provided a pharmaceutical composition for decreasing blood glucose levels comprising a therapeutic effective amount of a composition including an ethanol-water extract of Portulaca oleracea L. and a pharmaceutical acceptable carrier or diluent.

According to still a further aspect of the present invention there is provided a method of treating a hyperglycemia-related disease in a subject, the method comprising administering to a subject in need thereof a therapeutic effective amount of a composition including an ethanol-water extract of Portulaca oleracea L., thereby treating the hyperglycemia-related disease in the subject.

According to still a further aspect of the present invention there is provided a method of treating a hyperglycemia-related disease in a subject, the method comprising administering to a subject in need thereof a therapeutic effective amount of a composition including a polar fraction extract of Portulaca oleracea L., thereby treating the hyperglycemia-related disease in the subject.

According to still further features in the described preferred embodiments the polar extract is capable of lowering glucose levels in the blood.

According to still further features in the described preferred embodiments the polar fraction extract has Rf values in a range of 0.0-0.45 when subjected to thin-layer chromatographic fractionation on Silica Gel 60 F254 on aluminum using a solvent of dichloromethane: hexane: methanol in proportions of 1:1:0.2.

According to still further features in the described preferred embodiments the polar fraction extract has Rf values in a range of 0.0-0.32 when subjected to thin-layer chromatographic fractionation on Silica Gel 60 F254 on aluminum using a solvent of dichloromethane: hexane: methanol in proportions of 1:1:0.2.

According to still further features in the described preferred embodiments the polar fraction extract has Rf values in a range of 0.17-0.41 when subjected to thin-layer chromatographic fractionation on Silica Gel 60 F254 on aluminum using a solvent of dichloromethane: hexane: methanol in proportions of 1:1:0.2.

According to still further features in the described preferred embodiments the polar fraction extract of Portulaca oleracea L. is extracted by Soxlett extraction using methanol and has a Rf value selected from the group consisting of 0.0, 0.31, 0.34, 0.36, 0.39 and 0.45 when subjected to thin-layer chromatographic fractionation on Silica Gel 60 F254 on aluminum using a solvent of dichloromethane:hexane:methanol in proportions of 1:1:0.2.

According to still further features in the described preferred embodiments the polar fraction extract of Portulaca oleracea L. is extracted by Soxlett extraction using water and has a Rf value of 0.0 when subjected to thin-layer chromatographic fractionation on Silica Gel 60 F254 on aluminum using a solvent of dichloromethane:hexane:methanol in proportions of 1:1:0.2.

According to still further features in the described preferred embodiments the polar fraction extract of Portulaca oleracea L. is extracted with methanol and has a Rf value selected from the group consisting of 0.0, 0.15, 0.30 and 0.32 when subjected to thin-layer chromatographic fractionation on Silica Gel 60 F254 on aluminum using a solvent of dichloromethane:hexane:methanol in proportions of 1:1:0.2.

According to still further features in the described preferred embodiments the polar fraction extract of Portulaca oleracea L. is extracted with methanol:ethanol:water in proportions of 1:1:1 and has a Rf value selected from the group consisting of 0.17, 0.27, 0.30, 0.34, 0.39 and 0.41 when subjected to thin-layer chromatographic fractionation on Silica Gel 60 F254 on aluminum using a solvent of dichloromethane:hexane:methanol in proportions of 1:1:0.2.

According to still a further aspect of the present invention there is provided a method of treating a hyperglycemia-related disease in a subject, the method comprising administering to a subject in need thereof a therapeutic effective amount of a composition including a non-polar fraction extract of Portulaca oleracea L., thereby treating the hyperglycemia-related disease in the subject.

According to still further features in the described preferred embodiments the non-polar fraction extract of Portulaca oleracea L. is devoid of hydrocolloid.

According to still further features in the described preferred embodiments the hyperglycemia-related disease is selected from the group consisting of diabetes, Cushing's disease, Cushing's syndrome, eating disorders, impaired glucose tolerance, glomerular microangiopathy, diffuse glomerulosclerosis, nodular glomerulosclerosis, urinary infections, acute pyelonephritis, necrotizing papillitis, emphysematous pyelonephritis, glycogen nephrosis (armanni-ebstein lesion), retinopathy, nonproliferative retinopathy, capillary microaneurysms, retinal edema exudates, hemorrhages, proliferative retinopathy, proliferation of small vessels, hemorrhage fibrosis, retinal detachment, cataracts, transient refractive errors due to osmotic changes in lens, glaucoma due to proliferation of vessels in the iris, retinal infections, cerebrovascular atherosclerotic disease, neuropathy, skin infections, coronary atherosclerosis, myocardial infarction, peripheral atherosclerosis: limb ischemia, gangrene, increased fetal death rate, increased susceptibility to infection and delayed wound healing.

According to still further features in the described preferred embodiments the non-polar fraction extract has Rf values in a range of 0.11-0.89 when subjected to thin-layer chromatographic fractionation on Silica Gel 60 F254 on aluminum using a solvent of dichloromethane:hexane:methanol in proportions of 1:1:0.2.

According to still further features in the described preferred embodiments the non-polar fraction extract has Rf values in a range of 0.11-0.88 when subjected to thin-layer chromatographic fractionation on Silica Gel 60 F254 on aluminum using a solvent of dichloromethane:hexane:methanol in proportions of 1:1:0.2.

According to still further features in the described preferred embodiments the non-polar fraction extract has Rf values in a range of 0.17-0.91 when subjected to thin-layer chromatographic fractionation on Silica Gel 60 F254 on aluminum using a solvent of dichloromethane:hexane:methanol in proportions of 1:1:0.2.

According to still further features in the described preferred embodiments the non-polar fraction extract of Portulaca oleracea L. is extracted by Soxlett extraction using hexane and has a Rf value selected from the group consisting of 0.36, 0.45, 0.52, 0.71 and 0.88 when subjected to thin-layer chromatographic fractionation on Silica Gel 60 F254 on aluminum using a solvent of dichloromethane:hexane:methanol in proportions of 1:1:0.2.

According to still further features in the described preferred embodiments the non-polar fraction extract of Portulaca oleracea L. is extracted by Soxlett extraction using ethyl acetate and has a Rf value selected from the group consisting of 0.11, 0.18, 0.31, 0.36, 0.45, 0.52 and 0.71 when subjected to thin-layer chromatographic fractionation on Silica Gel 60 F254 on aluminum using a solvent of dichloromethane:hexane:methanol in proportions of 1:1:0.2.

According to still further features in the described preferred embodiments the non-polar fraction extract of Portulaca oleracea L. is extracted with hexane and has a Rf value selected from the group consisting of 0.3, 0.32, 0.41, 0.47 and 0.89 when subjected to thin-layer chromatographic fractionation on Silica Gel 60 F254 on aluminum using a solvent of dichloromethane:hexane:methanol in proportions of 1:1:0.2.

According to still further features in the described preferred embodiments the non-polar fraction extract of Portulaca oleracea L. is extracted with ethyl acetate and has a Rf value selected from the group consisting of 0.15, 0.36, 0.47, 0.73 and 0.89 when subjected to thin-layer chromatographic fractionation on Silica Gel 60 F254 on aluminum using a solvent of dichloromethane:hexane:methanol in proportions of 1:1:0.2.

According to still further features in the described preferred embodiments the non-polar fraction extract of Portulaca oleracea L. is extracted with hexane:DCM:ethyl-acetate in proportions of 1:1:1 and has a Rf value selected from the group consisting of 0.17, 0.30, 0.36, 0.41, 0.68 and 0.91 when subjected to thin-layer chromatographic fractionation on Silica Gel 60 F254 on aluminum using a solvent of dichloromethane:hexane:methanol in proportions of 1:1:0.2.

According to still a further aspect of the present invention there is provided a method of identifying agents for modulating glucose levels in the blood, the method comprising: (a) fractionating a Portulaca oleracea L. extract to thereby obtain a plurality of fractions; and (b) identifying from the plurality of fractions at least one fraction capable of modulating glucose levels in the blood, thereby identifying the agents for modulating glucose levels.

According to still further features in the described preferred embodiments fractionating is effected by employing a solvent gradient of increasing polarity

According to still further features in the described preferred embodiments step (b) is effected by testing an effect of the fraction on: (i) glucose adsorption through the intestines; and/or (ii) glucose transport into a cell.

The present invention successfully addresses the shortcomings of the presently known configurations by providing novel compositions derived from Portulaca oleracea L. and methods of using same for modulating blood glucose levels.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are graphs depicting the effect of Portulaca oleracea L. extract on blood glucose levels in non-insulin dependent diabetic patients with blood glucose levels greater than 300 mg/dl at the start of the trial (FIG. 1a) or in non-insulin dependent diabetic patients with blood glucose levels lower than 300 mg/dl at the start of the trial (FIG. 1b);

FIG. 2 is a bar graph depicting the effect of increasing concentrations of Portulaca oleracea L. extracts on viability of HepG2 cells, as determined using the MTT assay; All measurements were carried out in triplicates. Measurements were done on the same extract of Portulacea oleracea L.

FIG. 3 is a bar graph depicting the effect of increasing concentrations of Portulaca oleracea L. extracts on the viability of HepG2 and THP1 co-cultures, as determined using the MTT assay; Cell viability was measured as percentage of absorbance of treated and untreated cells. All measurements were carried out in triplicates.

FIG. 4 is a bar graph depicting the effect of increasing concentrations of Portulaca oleracea L. extracts on viability of LPS-stimulated HepG2 and THP1 co-cultures, as determined using the MTT assay;

FIG. 5 is a bar graph depicting the effect of increasing concentrations of Portulaca oleracea L. extracts on LDH release as a percentage of total LDH release from HepG2 cells (LDH concentration after complete cell destruction, releasing maximum quantity of LDH into the medium), as determined using the LDH assay; All measurements were carried out in triplicates.

FIG. 6 is a bar graph depicting the effect of increasing concentrations of Portulaca oleracea L. extracts on the percentage of LDH release from a co-culture of HepG2 and THP1 cells, as determined using the LDH assay; All measurements were carried out in triplicates.

FIG. 7 is a bar graph depicting the effect of increasing concentrations of Portulaca oleracea L. extracts on the percentage of LDH release from LPS-stimulated HepG2 and THP1 co-cultured, by the LDH assay.

FIG. 8 is a bar graph depicting the effect of increasing concentrations of Portulaca oleracea L. extracts on albumin production in HepG2 cells, as determined using the albumin secretion assay; Results are exhibited as percentage of total albumin secretion from cells from control untreated cells.

FIG. 9 is a bar graph depicting the effect of increasing concentrations of Portulaca oleracea L. alcohol-water extract on 10 μM FeSO₄-induced lipid peroxidation in sheep liver homogenate (n=3), using the MDA assay; X-axis depicts concentration of herbal extract (in mg/ml) in a solution of cell homogenate containing 10 μM iron sulfate. All measurements were carried out in triplicates.

FIG. 10 is a line graph depicting the effect of Portulaca oleracea L.extract on glucose adsorption through sheep intestine as a function of time; p1 and p2 are two different experiments effected under the same conditions.

FIG. 11 is a line graph depicting the effect of Portulaca oleracea L.extract on glucose uptake in yeast cells. The graph shows four separate experiments, and the average values. The measurement was performed 60 minutes following addition of the plant extract. Note, an increase in glucose uptake (expressed in %) is shown in the presence of increasing amounts of the plant extract.

FIG. 12 is a flow chart illustrating a step-wise procedure for purifying active components from plants.

### DESCRIPTION OF THE PREFERRED EMBODIMENTS

The present invention is of compositions derived from Portulaca oleracea L., which can be used for modulating blood glucose levels. Specifically, anti-hyperglycemic compositions of the present invention can be used to reduce blood glucose levels for treating hyperglycemia related diseases, such as Type II diabetes. Anti-hypoglycemic compositions of the present invention can be used to increase blood glucose levels for treating hypoglycemia related diseases, such as islet cell hyperplasia.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Type II diabetes is a chronic metabolic disease, which has a significant impact on the health, quality of life and life expectancy of patients. Though essential for improving glucose homeostasis, lifestyle management measures (i.e., exercise, diet and weight control) may be insufficient, rendering conventional drug therapies such as oral glucose lowering agents (i.e., hypoglycemic agents) necessary for many patients.

However, currently available orally administered anti diabetic drugs lose their effectiveness in a significant percentage of treated patients and in addition treatment is usually accompanied by adverse side effects such as weight gain, hypoglycemia, gastrointestinal disturbances, liver toxicity, high LDL cholesterol and high cost. For these reasons insulin is usually added to the oral agent at maximal oral delivery dosages. For these reasons, the medical field is constantly seeking new antihyperglycemic agents which can be used to treat type II diabetic patients.

Portulaca oleracea L., also known as Purslane, Verdolaga and Pursley, has been traditionally used as a remedy for a wide variety of ailments, in particular parasitic infections and digestive disorders. It has also been reported as a remedy for hyperglycemia. (i) PCT Appl. No. 00/00211 teaches the use of Portulacea oleracea L.-derived hydrocolloids for reducing sugar levels in the blood. (ii) Eskander and H. Won Jun (4) screened a number of herbs used in the Egyptian folk medicine, (including Portulacea oleracea L.) for the treatment of diabetes, to identify their hypoglycemic and hyperinsulinemic effects. Crude herb extracts generated as animal feed consisting of the dried ground herb suspended in water, were administered to alloxan diabetic rats. Eskander and H. Won Jun (4) showed that Portulaca oleracea L. is effective in reducing blood glucose levels of the treated rats. However, this activity could not be attributed to any particular plant component as the animal feed included solid particles of the plant as well as the water extract.

While reducing the present invention to practice, the present inventors uncovered that an ethanol-water extract of Portulaca oleracea L. can be efficiently used to reduce blood glucose levels in diabetic subjects, in a biosafe manner. While further reducing the present invention to practice, the present inventors uncovered that polar and non-polar fractions extracted from Portulaca oleracea L. are responsible for this anti-hyperglycemic effect.

As is illustrated in the Examples section which follows, administration of ethanol-water extract of Portulaca oleracea L. to human diabetic subjects with various abnormally high glucose levels (i.e., > or <300 mg/dl), significantly reduced their blood glucose levels to normal (see Example 2 of the Examples section). In an effort to isolate active components, which mediate this anti-diabetic effect, sequential polar extraction of the plant was effected (see Example 1 of the Examples section). Interestingly, both polar and non-polar components from Portulaca oleracea L. were accountable for reducing blood glucose levels, albeit with different mechanisms of action; while polar extracts from Portulaca oleracea L. increased glucose transport through the cell membrane into the cell, non-polar extracts decreased intestinal glucose adsorption.

Thus, the present findings show that non-polar and polar extracts from Portulaca oleracea L. (similarly to an ethanol-water extract of the same plant) act on different stages of glucose metabolism, essentially inhibition of glucose adsorption through the small intestines and promotion of glucose transport to cells, respectively, suggesting their individual or combined use in decreasing blood glucose levels.

The compositions of the present invention were shown to act in a biosafe manner as determined by in-vitro cell viability assays (i.e., MTT) and cell function assays (e.g., albumin secretion assay and lactate dehydrogenase assay, see Example 3), rendering their use clinically feasible.

Thus, according to one aspect of the present invention there is provided a composition of matter including an ethanol-water extract of Portulacea oleracea L.

Preferably the ratio of ethanol-water in the composition of this aspect of the present invention is 80% -20%.

The composition of this aspect of the present invention is capable of lowering glucose levels in the blood by increasing glucose transport into cells and/or decreasing glucose adsorption through the intestines.

As mentioned hereinabove, the present inventors were able to isolate active components (i.e., capable of decreasing blood glucose levels) from Portulaca oleracea L.

Thus according to another aspect of the present invention, there is provided a composition-of-matter including a polar fraction extract of Portulaca oleracea L tissue (e.g., leaves, stems, roots or whole plants).

As used herein the phrase “Portulaca oleracea L. tissue” refers to an intact Portulaca oleracea L. plant or parts thereof, such as leaves, roots, stems, exudate and the like.

As used herein a “polar fraction extract” refers to a Portulaca oleracea L. extract which is composed of polar components and obtained using a polar solvent such as, for example, alcohol. The Portulaca oleracea L. extract of the present invention is preferably devoid of solid plant particles.

The term “polar” refers to compounds that have polar functional groups (e.g. Amides, Acids, Alcohols, Ketones, Aldehydes, Amines), which have negative and positive poles forming a dipole moment. The polar fraction extract may include compounds which are found on the plant tissue (i.e., exudates), within the plant tissue but outside of the plant cells (apoplast), or within the cells of the plant (cytoplasmic, vacuolar or organelle sequestered).

Examples of solvents suitable for polar fraction extraction include, but are not limited to water and alcohols, such as for example, methanol, ethanol, and isopropyl alcohol. Whole plant tissue can be immersed in such solvents without need for tissue disintegration although such physical manipulation of the tissue is preferred since it substantially improves extraction of polar components. Examples of tissue disintegration techniques which can be utilized with the present invention include grinding of frozen tissue and homogenization using a homogenizer. Other methods of pretreating for improving extraction of the plant tissue are described hereinbelow.

As is illustrated in the Examples section which follows, the polar fraction extract generated according to the teachings of the present invention is capable of lowering glucose levels in the blood to normal levels [i.e., normal fasting blood (i.e., plasma) glucose levels less than 115 mg/dl]. As is illustrated in Example 4 of the Examples section, analysis conducted on the polar extract revealed that it includes components which are capable of increasing glucose transport into cells.

The composition of this aspect of the present invention includes components having retention factor (Rf, the distance traveled by the compound divided by the distance traveled by the solvent front) values in a range of 0.0-0.50, more preferably in a range of 0.0-0.45, even more preferably in a range of 0.0-0.32 and yet even more preferably in a range of 0.17-0.41, when subjected to thin layer chromatography fractionation on Silica Gel 60 F254 on aluminum sheets [20×20 cm, (Merck KGaA, Darmstadt, Germany)] using a solvent mixture of dichloromethane:hexane:methanol in proportions of 1:1:0.2.

The polar fraction extract of Portulaca oleracea L. can be extracted via Soxlett extraction (see Example 1 below) using methanol, in which case it includes components having Rf values of 0.0, 0.31, 0.34, 0.36, 0.39 and 0.45 when subjected to thin-layer chromatographic fractionation on Silica Gel 60 F254 on aluminum using a solvent of dichloromethane:hexane:methanol in proportions of 1:1:0.2.

The polar fraction extract of Portulaca oleracea L. of the present invention can be extracted by Soxlett extraction using water in which case it includes components having Rf values of 0.0 when subjected to thin-layer chromatographic fractionation on Silica Gel 60 F254 on aluminum using a solvent of dichloromethane:hexane:methanol in proportions of 1:1:0.2.

The polar fraction extract of Portulaca oleracea L. of the present invention can be extracted with methanol in which case it includes components having Rf values of 0.0, 0.15, 0.30 and 0.32 when subjected to thin-layer chromatographic fractionation on Silica Gel 60 F254 on aluminum using a solvent of dichloromethane:hexane:methanol in proportions of 1:1:0.2.

Finally, the polar fraction extract of Portulaca oleracea L. can be extracted with methanol:ethanol:water in proportions of 1:1:1 in which case it includes components having Rf values of 0.17, 0.27, 0.30, 0.34, 0.39 and 0.41 when subjected to thin-layer chromatographic fractionation on Silica Gel 60 F254 on aluminum using a solvent of dichloromethane:hexane:methanol in proportions of 1:1:0.2.

As is shown in Example 1 of the Examples section which follows, non-polar extracts from Portulaca oleracea L. are capable of reducing glucose absorption through the small intestines and as such can be used to reduce glucose concentration in the blood.

Thus, according to yet another aspect of the present invention, there is provided a composition-of-matter including a non-polar fraction extract of Portulaca oleracea L tissue.

As used herein a “non-polar fraction extract” refers to a Portulaca oleracea L. extract which is composed of non-polar components and obtained using a non-polar solvent.

The term “non-polar” refers to compounds that have non-polar functional groups (e.g., alkyl, cyano, ester, and other non-ionic groups), which have no separation of charge, such that no positive or negative poles are formed. Examples of non polar solvents which can be used for extracting non-polar components include, but are not limited to, hexane, cyclohexane, ethyl acetate and toluene.

The non-polar fraction extract may include compounds which are found on the plant tissue (i.e., exudates), within the plant tissue but outside of the plant cells (apoplast), or within the cells of the plant (cytoplasmic, vacualar or organelle sequestered).

The non-polar fraction extract of this aspect of the present invention is capable of decreasing glucose levels in the blood, preferably to normal levels [e.g., at least 75 mg/dl] suggesting use thereof as anti-hyperglycemic agent (i.e., capable of decreasing blood sugar levels). As is illustrated in Example 1 of the Examples section which follows, the non-polar extract of the present invention decreases glucose adsorption through the small intestines and as such can be used as anti hyperglycemic agent.

The composition of this aspect of the present invention includes components having Rf values in a range of 0.11-0.95, more preferably in a range of 0.11-0.89, even more preferably in a range of 0.11-0.88 and yet even more preferably in a range of 0.17-0.91, when subjected to thin layer chromatography fractionation on Silica Gel 60 F254 on aluminum plate [20×20 cm, (Man. Merck KGaA, Darmstadt, Germany)] using a solvent of dichloromethane:hexane:methanol in proportions of 1:1:0.2.

Preferably, the non-polar fraction extract of Portulaca oleracea L. is extracted by Soxlett extraction (see below) using hexane and includes components having Rf values of 0.36, 0.45, 0.52, 0.71, or 0.88 when subjected to thin-layer chromatographic fractionation on Silica Gel 60 F254 on aluminum using a solvent of dichloromethane:hexane:methanol in proportions of 1:1:0.2.

The non-polar fraction extract of Portulaca oleracea L. of the present invention can be extracted via Soxlett extraction using ethyl acetate in which case it includes components having Rf values of 0.11, 0.18, 0.31, 0.36, 0.45, 0.52 or 0.71 when subjected to thin-layer chromatographic fractionation on Silica Gel 60 F254 on aluminum using a solvent of dichloromethane:hexane:methanol in proportions of 1:1:0.2.

The non-polar fraction extract of Portulaca oleracea L. of the present invention can be extracted with hexane in which case it includes components having Rf values of 0.3, 0.32, 0.41, 0.47 or 0.89 when subjected to thin-layer chromatographic fractionation on Silica Gel 60 F254 on aluminum using a solvent of dichloromethane:hexane:methanol in proportions of 1:1:0.2.

The non-polar fraction extract of Portulaca oleracea L. of the present invention can be extracted with ethyl acetate in which case it includes components having Rf values of 0.15, 0.36, 0.47, 0.73 or 0.89 when subjected to thin-layer chromatographic fractionation on Silica Gel 60 F254 on aluminum using a solvent of dichloromethane:hexane:methanol in proportions of 1:1:0.2.

Finally, the non-polar fraction extract of Portulaca oleracea L. can extracted with hexane:dichloromethane(DCM):ethyl acetate: in proportions of 1:1:1 in which case it includes components having Rf values of 0.17, 0.30, 0.36, 0.41, 0.68 or 0.91 when subjected to thin-layer chromatographic fractionation on Silica Gel 60 F254 on aluminum using a solvent of dichloromethane:hexane:methanol in proportions of 1:1:0.2.

Anti-hyperglycemic agents of the present invention (i.e., polar and non-polar fraction extracts) are preferably devoid of hydrocolloid (a substance that forms a gel in an aqueous solution).

Anti-hyperglycemic agents of the present invention can be isolated from Portulaca oleracea L. by extracting polar components from the plant.

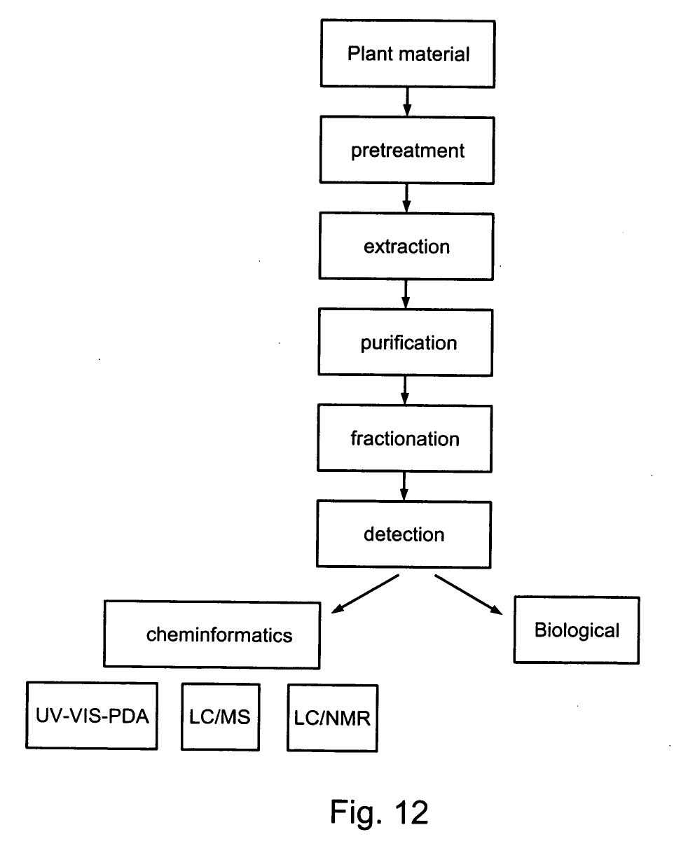

Methods of isolating active components from plants are well known in the art. A general isolation procedure of active components from plant material is described in the flow diagram of FIG. 12 [see also Wink (1999) Function of Plant Secondary Metabolites and their Exploitation in Biotechnology. CRC Press].

Typically, plant tissue of interest is obtained. It will be appreciated that plant organelles may also be used as a source of the polar components. For example, plant vacuoles may be used for extracting polar components. Methods of isolating plant protoplasts as a source of vacuoles are well known in the art [see e.g., Guy, M., et al., Plant Physiol. 64:61-64 (1979)].

The plant tissue, thus obtained may be fresh or dried. Preferably, dried material is used for simplifying further large-scale extraction. Thus, plant tissue may be subjected to pre-treatment such as drying (see also Example 1 of the Examples section) and grinding which may prolong storage thereof prior to use.

Plant material is then subjected to extraction to isolate the polar components described herein (active ingredients).

As used herein the term “extraction” refers to the procedure of separating mixtures based on chemical and/or physical differences in features such as solubility in polar versus non-polar solvents. Numerous extraction procedures are known in the art.

Extraction of polar fractions from Portulaca oleracea L. according to this aspect of the present invention is preferably effected sequentially using solvents of increasing polarity. Essentially, dried plant material is subjected to non-polar extraction, either by separate solvents (e.g., hexane followed by ethyl acetate and chloroform) or by using a mixture of such solvents. Thereafter, the same plant material is subjected to polar extraction. Such sequential extraction steps improves the resolution of isolating active components.

For example, Soxlett extraction of polar components effected by employing a solvent gradient of increasing polarity (e.g., hexane, dichloromethane (DCM), ethanol and water) is shown in Example 1 of the Examples section which follows. This method is preferably used with thermostable compounds.

Active components (e.g., polar components) are then purified from the extract using purification methods, which are well known in the art. For example, extracts can be separated on silica gel (TLC, thin layer chromatography) plates with solvents of increasing polarity. Samples are preferably applied immediately on the plate and run without delay to minimize oxidation, though drying of the extract may also be effected (see Example 1 of the Examples section which follows). The active components are visualized under UV light. Visualization may also be effected by spraying the plate with a developer (e.g., Phosphomolybdic Acid solution in ethanol) and heating to allow for the development of color changes. This TLC method is efficient, rapid and combines sensitivity and simplicity with low cost.

Efficient extraction of active components from powdered plant material can also be performed by accelerated solvent extraction (ASE), a procedure which utilizes enhanced solubilization kinetics at elevated temperature and pressure [Obana Analyst. (1997) 122 (3):217-20].

Active components may be qualified using biological assays. Selection of a suitable biological readout depends on the desired function of the active component. Thus, to identify compounds, which modulate blood glucose levels, any of the assays described in Example 4 may be used. Active ingredients are chemically identified using chemoinformatics as further described hereinbelow.

For example, HPLC coupled to UV photodiode array detection (LC/DAD-UV) and to mass spectrometry (LC/MS or LC/MS/MS) can provide structural information on the active components in the extract prior to isolation [see Outtara Phytochemistry (2004) 65 (8):1145-51]. Chemical screening using hyphenated techniques such as LC/UV, LC/MS and LC/MS/NMR [Mazza J AOAC Int. 2004 Janurary-Feburary; 87 (1):129-45; Wolfender J Chromatogr A. 2003 Jun. 6; 1000 (1-2):437-55] can provide structural information of known plant constituents with minute amounts of plant material. This enables the differentiation between novel compounds and known compounds directly from crude plant extracts.

Alternatively, plant extracts which exhibit desired activity (e.g., anti-hyperglycemic) in the bioassay (e.g., glucose transport through the small intestines) can be chemically screened by analysis using LC/UV/MS. Separation may be performed in reversed phase RP-C₁₈ column with broad acetonitrile or methanol gradients. UV spectra are recorded and molecular weight information is obtained by MS with thermospray, continuous-flow fast atom bombardment, atmospheric pressure chemical ionization or electrospray ionization. Fragment information is obtained by tandem MS/MS or multiple stage MSₙ experiments while LC/NMR is used for confirmation of compound identity.

Integrated, information rich detection systems such as chromatography-ultraviolet nuclear magnetic resonance-mass spectrometry (LC-UV-NMR-MS), and other genomics and proteomics approaches can be used to identify active components of interest from the plant.

Isolation of non-polar components from the plant (i.e., Portulaca oleracea L.) can be effected using the methods described above, only non-polar solvents are used in the first step of non-polar component extraction.

As mentioned hereinabove, anti-hyperglycemic agents of the present invention (i.e., polar, non-polar, ethanol-water extracts) can be used for modulating glucose levels in the blood.

Thus, the present invention envisages a method of treating a hyperglycemia-related disease in a subject.

As used herein the phrase “hyperglycemia-related disease” refers to a disease which is dependent on hyperglycemia for its onset and/or progression. As used herein the term “hyperglycemia” refers to abnormally high glucose concentration in the blood (i.e., >115 mg/dl). Examples of hyperglycemia-related diseases and disorders include, but are not limited to, diabetes, Cushing's disease, Cushing's syndrome, eating disorders (e.g., anorexia nervosa, anorexia bulimia), impaired glucose tolerance (IGT), glomerular microangiopathy, diffuse glomerulosclerosis, nodular glomerulosclerosis (Kimmel-stiel-Wilson disease), urinary infections, acute pyelonephritis, necrotizing papillitis, emphysematous pyelonephritis, glycogen nephrosis (armanni-ebstein lesion), retinopathy, nonproliferative retinopathy, capillary microaneurysms, retinal edema exudates, hemorrhages, proliferative retinopathy, proliferation of small vessels, hemorrhage fibrosis, retinal detachment, cataracts, transient refractive errors due to osmotic changes in lens, glaucoma due to proliferation of vessels in the iris, retinal infections, cerebrovascular atherosclerotic disease: strokes, peripheral neuropathy; peripheral sensory and motor cranial, autonomic, skin infections: folliculitis leading to carbuncles, necrobiosis lipoidica diabeticorum: due to microangiopathy, xanthomas: secondary to hyperlipidemia, coronary atherosclerosis: myocardial infarction, peripheral atherosclerosis: limb ischemia, gangrene, increased fetal death rate (placental disease, neonatal respiratory distress syndrome, infection), increased susceptibility to infection and delayed wound healing.

As used herein the term “treating” refers to preventing, curing, reversing, attenuating, alleviating, minimizing, suppressing or halting the deleterious effects of the diseases of the present invention.

As used herein the term “subject in need thereof” refers to a mammalian subject (e.g., human) suffering from the disease of the present invention or is predisposed thereto.

The method according to this aspect of the present invention is effected by administering to the subject (further described hereinbelow), a therapeutically effective amount of a composition of the present invention (i.e., polar-, non-polar-, or ethanol-water extract of Portulaca oleracea L. described above), to thereby treat the hyperglycemia-related disease in the subject. It will be appreciated that since each of the compositions of the present invention (i.e., polar and non-polar extracts) acts to inhibit different stages of glucose metabolism and as such complement inhibition of glucose metabolism, combined administration thereof is preferable to improve therapeutic efficacy. These compositions can thus be administered to the subject simultaneously (together) or sequentially.

Compositions (i.e., anti-hyperglycemic agents) of the present invention may be administered to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art. Preferably, compounds of the present invention are orally administered. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that the above-described therapeutic procedures of the present invention may be combined with other treatment modalities known in the art. For example, the anti-hyperglycemic agents of the present invention may be combined with a variety natural or synthetic substances in methods of treating diabetes. Examples of such substances include, but are not limited to gymnema sylvestre, fenugreek, bitter melon, α-lipoic acid, banaba Leaf, yacou root, momordica charantia, olive leaf extract, pterocarpus marsupium, salacia reticulate, garlic, hawthorn, corosolic acid, ursolic acid, D-pinitol, aloe vera, chromium picolinate, phosphatidylserine, omega 3 fatty acids, resistant starch, catharanthus roseus, anacardium occidentale, syzygium cumini, eucalyptus globules, lupinus albus, allium cepa, allium sativum, tecoma stans, urtica dioica, taraxacum officinale, kyllinga monocephala, phyllanthus emblica, phyllanthus niruri, azadirachta indica, morbus alba, poterium ancistroides, daucus carota, insulin and other orally administered agents such as sulfonylureas, biguanides, α-glucosidase inhibitors, thiazolidinediones and meglitinides. These combinations of substances for use in methods of treating diabetes provide the benefits attributable to each component (i.e., the Portulaca oleracea L. extract and the substance with which it is combined for administration).

It will be appreciated that compositions of the present invention may also be used as nutritional additives, such as for improving sports nutrition. Accordingly, the compositions of the present invention may be used alone or combined with an effective dose of any one of the following naturally occurring (e.g., plant-based) substances or chemical compounds: creatine, creatine monohydrate, creatine salts such as creatine citrate, creatine pyruvate, creatine derivatives and salts thereof, phosphocreatine, caffeine, α-lipoic acid, glucosamine, chondroitin, hydrolyzed collagen, methylsulfonyl-methane, whey protein, L-glutamine, phosphatidylcholine, choline, choline salts, phosphatidylserine, beta-hydroxy beta-methylbutyrate, pyruvate, L-camitine, D-ribose, an amino acid (a conventional amino acid), a branched chain amino acid, S-adenosylmethionine, taurine, conjugated linoleic acid, α-lipoic acid, α-lipoic acid salts, and glycerin. In referencing the salts of various compounds, the invention contemplates the compound and any suitable salt-forming counterions (such as alkali metal ions, alkaline earth metal ions, halogen ions, organic cations, organic ions, complex ions and any other counterion known in the art).

It will be further appreciated that compositions of the present invention may also be used for modulating body mass (i.e., weight control). In this case, an effective dose of a composition of the present invention is combined with an effective dose of any one of the following naturally occurring (e.g., plant-based) substances or chemical compounds: pyruvate, L-carnitine, hydroxycitric acid, ephedrine, caffeine, and conjugated linoleic acid (CLA). These combinations of substances for use in methods for improving nutrition, such as sports nutrition, also provide the benefits attributable to each component (i.e., the Portulaca oleracea L. extract and the substance with which it is combined for administration).

Compositions of the present invention can be packed in a therapeutic or a nutritional kit.

For example, compositions of the present invention can be packaged in one or more containers with appropriate buffers and preservatives and used for directing therapeutic treatment.

Thus, the compositions (e.g., ethanol-water, polar and non-polar fraction extracts of Portulaca oleracea L.) can be mixed in a single container or placed in individual containers. Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

The kit can also include instructions for determining if the tested subject is suffering from, or is at risk of developing, a condition, disorder, or disease associated abnormal blood glucose level.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Purification, Isolation, and Identification of Bioactive Components from Portulaca oleracea L. (Portulaca oleracea L.)

In initial experiments, the extraction of Portulaca oleracea L. was effected in stages, using solvents that vary from non-polar to polar. Two main preliminary procedures were developed and compared for efficacy. Results obtained thereby were then utilized in the development of a standard extraction procedure.

### Example 1a

Repeated Soxlett Extraction

Experimental Procedure

Materials—Solvents were purchased from Frutarom Ltd. Haifa, ISRAEL or from J. T. Baker, Deventer Holland and were analytical grade unless otherwise specified. D-glucose monohydrate was purchased from Riedel-de Haen, Seelze Germany. Additional research chemicals were purchased from Sigma-Aldrich, Milwaukee, U.S.A.

Soxlett extraction—20 g of ground, dried Portulaca oleracea L. material were placed in a standard Soxlett extraction thimble and the material was extracted with 250 ml of solvent as further described below. Extraction was effected five times, each time using one of the following five different solvents of increasing polarity: hexane, ethyl acetate, dichloromethane (DCM), methanol, and water. Extraction was continued for two full cycles (i.e., the thimble was filled with solvent and emptied twice) under reflux of the solvent. Following extraction, the solvent was evaporated in a vacuum rotavapor and the dried extract was collected and weighed. The residual plant material was dried in a drying oven at 65° C. prior to additional extraction. The dry extracts were collected and analyzed by thin layer chromatography [(TLC), Silica plates, Silica Gel 60 F254 on aluminum, in Dichloromethane (DCM)-Hexane-Methanol, 1:1:0.2]. The TLC plates were visualized by UV and by staining with 5% phosphomolybdic acid in IPA.

Glucose Adsorption through the Intestines—See Example 4 below.

Glucose Transport into the Cell—See Example 4.

Results

Extraction yields—Table 1 below displays the results of the Repeated Soxlett Extraction of Portulaca oleracea L. using each of the five solvents as well as the residue, listing the corresponding weight of each extract in grams, the percentage extracted of the total amount of plant material, and remarks on physical characteristics.

TABLE 1

| | | Percentage | |
|---|---|---|---|
| Extract | Weight (g) | of Total | Remarks |
| Hexane | 0.48 | 2.40 | powder |
| Ethyl Acetate | 0.40 | 2.00 | solid material |
| Dichloromethane | 0.10 | 0.50 | sticky material |
| Methanol | 1.39 | 6.95 | sticky material |
| Water | 2.38 | 11.90 | sticky material |
| Residue | 15.1 | 75.5 | plant material |

TLC analysis—Table 2 below lists the results of TLC analysis of each of the five Repeated Soxlett extracts. All spots visible by UV and/or by staining are reported as Rf values.

TABLE 2

| | Rf Value | | | | | | | | | | |
|---|---|---|---|---|---|---|---|---|---|---|---|
| Extract | 0.88 | 0.71 | 0.52 | 0.45 | 0.39 | 0.36 | 0.34 | 0.31 | 0.18 | 0.11 | 0.0 |
| Hexane | X | X | X | X | | X | | | | | |
| Ethyl Acetate | | X | X | X | | X | | X | X | X | |
| Dichloromethane | | | X | X | | X | X | X | X | | |
| Methanol | | | | X | X | X | X | X | | | X |
| Water | | | | | | | | | | | X |

Analysis of the TLC patterns obtained showed that the most intensive spots (0.45 and 0.36) appeared to some extent in four of the five fractions. The intensity of the spots appeared to be strongest in the DCM extract, although this may be misleading, since DCM is a very volatile solvent which may have evaporated prior to being applied onto the TLC plate resulting in a smaller and more concentrated spot. The separation obtained in this TLC analysis indicated that many compounds appeared to have both polar and non-polar properties and thus may be extracted under both conditions, as is the case for molecules such as fatty acids, steroids, and amines.

Analysis of Biological Activity—Extracts were analyzed for biological activity using two standard bioassays: glucose adsorption through the intestine, in which the effect of the extract on the adsorption of glucose through the intestine is measured, as described in Example 4, and glucose transport into the cell, in which the effect of the extract on the transport of glucose from the bloodstream to the cell interior is measured, as described as well in Example 4. Table 3 below lists the results of the bioassays of each of the five extracts.

TABLE 3

| | | Ethyl | Dichloro- | | |
|---|---|---|---|---|---|
| Bioassay | Hexane | Acetate | methane | Methanol | Water |
| Decreased | positive | positive | negative | negative | negative |
| Intestinal | | | | | |
| glucose | | | | | |
| Adsorption | | | | | |
| Increased | negative | negative | negative | positive | positive |
| Glucose | | | | | |
| Transport | | | | | |
| through cell | | | | | |
| membrane | | | | | |
| into the cell | | | | | |

### Example 1b

Extraction of Portulaca oleracea L. Active Components by the Room Temperature (RT) Method

Materials and Experimental Procedures

RT extraction—100 grs of dried, ground Portulaca oleracea L. material were placed in a glass beaker with 1 l of solvent and stirred at room RT for 24 hours (hrs). Extraction was effected five times in parallel using one of five different solvents of increasing polarity: hexane, ethyl acetate, DCM, methanol, and water. Following extraction, the plant material was filtered out, the solvent of the filtrate was evaporated in a vacuum rotavapor, and the dried extract was collected and weighed. The residual plant material was dried in a drying oven at 65° C. prior to additional extraction. The dry extracts were collected and analyzed by TLC, as described in Example 1a.

Glucose Adsorption through the Intestines—See Example 4 below.

Glucose Transport into the Cell—See Example 4.

Results

Extraction yields—Table 4 below displays the results of the RT Extraction using each of the five solvents as well as the residue, listing the corresponding weight of each extract in grams, the percentage extracted of the total amount of plant material, and remarks on physical characteristics.

TABLE 4

| | | Percentage | |
|---|---|---|---|
| Extract | Weight (g) | of Total | Remarks |
| Hexane | 0.41 | 0.41 | Dry Powder |
| Ethyl Acetate | 0.55 | 0.55 | Dry Powder |
| Dichloromethane | 0.2 | 0.2 | Dry Powder |
| Methanol | 5.4 | 5.4 | Amorphous powder |
| Water | 7.0 | 7.0 | Dry Powder |
| Residue | 87.0 | 87.0 | Plant material |

TLC analysis—Table 5 lists the results of TLC analysis of each of the five RT extracts. All spots visible by UV and/or by staining are reported as Rf values.

TABLE 5

| | Rf Value | | | | | | | | |
|---|---|---|---|---|---|---|---|---|---|
| Extract | 0.89 | 0.73 | 0.47 | 0.41 | 0.36 | 0.32 | 0.30 | 0.15 | 0.0 |
| Hexane | X | | X | X | | X | X | | |
| Ethyl Acetate | X | X | X | | X | | | X | |
| Dichloromethane | X | X | | X | | X | X | X | |
| Methanol | | | | | | X | X | X | X |
| Water | | | | | | | | | X |

Analysis of the TLC pattern showed a clear separation between spots in the higher region (0.89-0.47) and spots in the lower region (0.30-0.0). The non-polar to polar transition appeared to be more pronounced than in the Soxlett extraction where the spots were present throughout he whole spectrum from 0-1

These results indicate removal of most of the non-polar material by hexane and extraction of the polar material using methanol or water. The large fractions in the middle region of the TLC plate indicate the presence of molecules with both polar and non-polar characteristics.

Analysis of Biological Activity—The extracts were analyzed for biological activity using two standard bioassays: glucose adsorption through the intestine and glucose transport into the cell, as described as well in Example 4. Table 6 lists the results of the bioassays of each of the five extracts.

TABLE 6

| | | Ethyl | | | |
|---|---|---|---|---|---|
| Bioassay | Hexane | Acetate | Dichloromethane | Methanol | Water |
| Decreased | positive | positive | negative | negative | negative |
| Intestinal | | | | | |
| glucose | | | | | |
| Adsorption | | | | | |
| Increased | negative | negative | negative | positive | positive |
| Glucose | | | | | |
| Transport | | | | | |
| through cell | | | | | |
| membrane | | | | | |
| into the cell | | | | | |

### Example 1c

Comparison of the Repeated Soxlett and RT Extraction Methods

The two preliminary extraction methods were compared based upon the following criteria: efficiency of extraction, i.e., the percentage of plant material extracted using each solvent and the concentration of extracted material in the solvent in g/ml; cleanliness of extraction, i.e., the formation of streaks in TLC indicating decomposition of material during extraction; materials extracted with each solvent in both procedures detected by TLC; and bioactivity of the extracts.

Efficiency of extraction—The overall data obtained from both extraction procedures is similar (see Tables 1-5 above) and in general, the different solvents of both procedures gave rise to identical TLC spots, indicating probable extraction of similar components. The Repeated Soxlett extraction resulted in higher yields of material than the RT extraction (19% versus 12%, respectively, using polar solvents and 5% versus 1.2%, respectively, using non-polar solvents). Whether this indicates a higher efficiency of the Soxlett extraction or the extraction of undesired plant materials is currently under investigation.

Cleanliness of extraction—The RT extraction was found to be cleaner than the Repeated Soxlett extraction, where streaked and elongated spots on TLC plates were observed, indicating decomposition products as a result of excessive heating.

TLC comparison—In general both extraction procedures yielded similar major TLC spots with similar intensities. The Repeated Soxlett procedure yielded more spots, which may be a result of higher concentrations of various materials or of decomposition.

Bioactivity—The bioactivity detected was clearly distributed over the polar-non-polar divide. While glucose transport into the cell was clearly associated with polar fractions (methanol and water) and no activity was found in the non-polar fractions, glucose adsorption through the intestinal wall was associated with non-polar components; Glucose adsorption was clearly reduced by the non-polar fractions.

Discussion

The above-described extraction procedures exhibited the presence of two separate plant fractions each of which is active in a different part of the metabolism of glucose. The non-polar extract fraction exhibited decreased glucose adsorption activity through the intestine, while the polar fraction exhibited enhanced glucose transport to the cells. These fractions are a stage on the way to elucidating the active ingredients

Clearly at least two and possibly three different factors are responsible for the observed biological activities of the Portulaca oleracea L. extract.

The extraction procedure may be simplified by reducing the extractions to two steps, one using non-polar solvents and one using polar solvents. In addition, although the yield of RT extraction is somewhat lower, this method is preferable since possible decomposition may be avoided. Whether additional important compounds are extracted by the Repeated Soxlett method remains to be investigated. Note that sequential extraction procedure should be employed to maximize isolation of active components, essentially, a nonpolar extraction must be performed which is followed by a polar extraction.

Example 1d

Extraction of Portulaca oleracea L. by the Standard Two-Step Method

Materials and Experimental Procedures

Standard Two-Step extraction—200 g of ground, dried Portulaca oleracea L. material was placed in a glass beaker with 2 l of a solvent mixture and stirred at RT for 24 hrs. The extraction was sequentially effected with two different solvent mixtures:non-polar:hexane-DCM-ethyl acetate, 1:1:1, and polar:methanol-ethanol-water, 1:1:1. Following extraction, the plant material was filtered out, the solvent of the filtrate was evaporated in a vacuum rotavapor, and the dried extract was collected and weighed. The residual plant material was dried in a drying oven at 65° C. prior to additional extraction. The dry extracts were collected and analyzed by TLC, as described in Example 1a.

Glucose Adsorption Through the Intestines—See Example 4 below.

Glucose Transport Into the Cell—See Example 4.

Results

Extraction yields—Table 7 below displays the weight of each fraction (non-polar, polar, or residue) of the Standard Two-Step extraction in grams, the corresponding percentage extracted of the total amount of plant material, as well as remarks on physical characteristics.

TABLE 7

| | | Percentage | |
|---|---|---|---|
| Extract | Weight (g) | of Total | Remarks |
| Non-polar | 1.68 | 0.84% | Dry Powder |
| Polar | 21.6 | 10.8% | Dry Powder |
| Residue | 175 | 88% | Plant material |

TLC analysis—Table 8 displays the results of TLC analysis, reported as Rf values, of all spots visible under UV and by staining.

TABLE 8

| | Rf Value | | | | | | | |
|---|---|---|---|---|---|---|---|---|
| Extract | 0.91 | 0.68 | 0.41 | 0.36 | 0.34 | 0.30 | 0.27 | 0.17 |
| Non-polar | X | X | X | X | | X | | X |
| Polar | | | X | | X | X | X | X |

Three TLC spots, 0.41, 0.30 and 0.17, were common to both extraction procedures. Further analysis is required to show any correlated biological activity.

Analysis of Biological Activity—The extracts were analyzed for biological activity using the two standard bioassays: glucose adsorption through the intestine and glucose transport to the cell, as described in Example 4. Table 9 summarizes the biological activities of the non-polar and polar fractions of Portulaca oleracea L. extract, measured using the two bioassays.

TABLE 9

| Bioassay | Non-polar | Polar |
|---|---|---|
| Decreased Intestinal glucose adsorption | Positive | negative |
| Increased Glucose transport through cell | negative | Positive |
| membrane into the cell | | |

Discussion

A standard extraction procedure, aimed at identification and purification of the bioactive components of Portulaca oleracea L., has been developed. This two-step procedure, which consists of a non-polar extraction followed by a polar extraction, resulted in a good initial separation of both polar and non-polar fractions as well as of bioactive components. The Two-Step extraction of Portulaca oleracea L. forms a good basis for a further purification and identification of the bioactive factors.

### Example 2

### Effects of Portulaca oleracea L. Extracts on Blood Glucose Levels of Human Diabetic Patients

Materials and Experimental Procedures

Trial Subjects—The trial on human subjects was conducted in cooperation with physicians and biochemical laboratories in the Nazareth region of Israel. Two groups of non-insulin (Type 2) diabetic patients were evaluated for the effect of Portulaca oleracea L. extract on levels of glucose in the blood. Patients were assigned to one of two groups on the basis of the initial level of glucose in the blood. At the start of the trial, the blood glucose levels in the patients of the first group were greater than 300 mg/dl, while those of the second group were less than 300 mg/dl. Table 10 below displays the characteristics and clinical histories of patients with greater than 300 mg/dl of blood glucose at the start of the trial.

TABLE 10

| | | | | | Presence | Years | |
|---|---|---|---|---|---|---|---|
| | Patient | Weight | | | of other | Since | Glucose |
| No | Initials | (kg) | Age | Sex | conditions | Diagnosis | Levels |
| 1 | N. T. | 83 | 57 | F | — | 16 | 300 |
| 2 | S. R. | 95 | 64 | M | High blood | 11 | 455 |
| | | | | | pressure | | |
| 3 | M. T. | 89 | 62 | M | — | 7 | 380 |
| 4 | M. A. | 92 | 59 | M | High | 10 | 310 |
| | | | | | Cholesterol | | |
| 5 | F. J. | 75 | 55 | F | — | 3 | 320 |

Table 11 below displays the characteristics and clinical histories of patients with less than 300 mg/dl of blood glucose at the start of the trial.

TABLE 11

| | | | | | | | glucose |
|---|---|---|---|---|---|---|---|
| | | | | | Presence | Years | levels at |
| | Patient | Weight | | | of other | Since | start of |
| No | Initials | (kg) | Age | Sex | conditions | Diagnosis | trial |
| 1 | A. A. | 82 | 47 | M | — | 5 | 225 |
| 2 | B. R. | 85 | 67 | F | — | 8 | 250 |
| 3 | A. H. | 107 | 58 | F | High blood | 3 | 190 |
| | | | | | pressure | | |
| 4 | G. G. | 96 | 72 | M | High | 14 | 205 |
| | | | | | Cholesterol | | |

Preparation of Portulaca oleracea L. extract for patient administration—An ethanol-water (80%-20%) Portulaca oleracea L. extract was produced with a TCP binder (β-tri-calcium phosphate). The extraction was performed at 40° C. for 4 hours using ground dried plant material and an ethanol-water mixture (80-20) at a 10-90 (w/w) ratio. The solution was filtered and the extract dried in vacuum at 0° C. in the presence of TCP, a commercially used binder material.

The final composition of the product was 25% extract and 75% binder material. The yield of the extraction was 8-9% by weight.

Measurement of blood glucose levels—Glucose was measured on a weekly basis following 12 hrs of fasting using commercially available glucometers.

Results

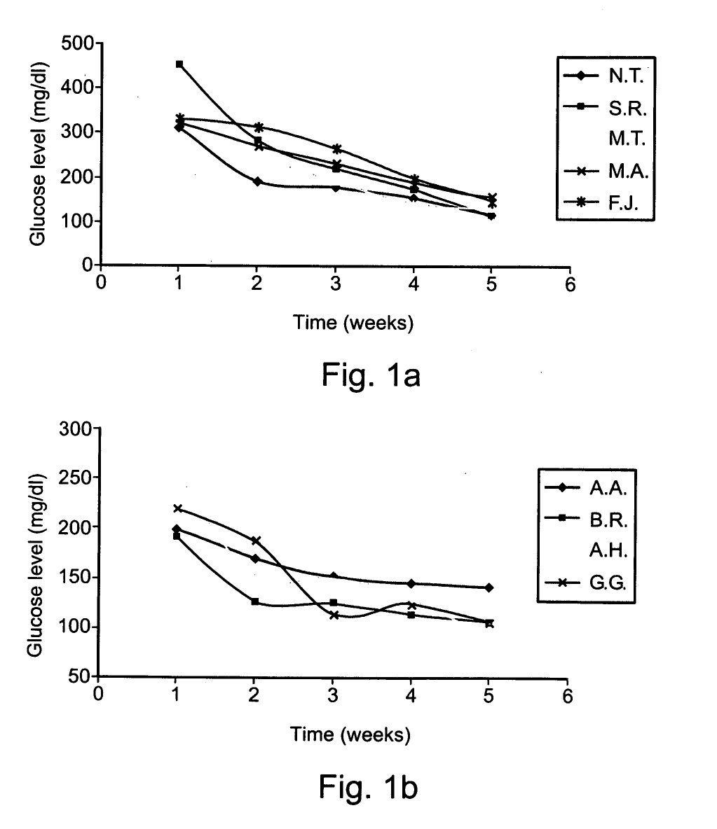

Portulaca oleracea L. ethanol water extract (dry powder encapsulated in standard vegetable gelatin capsules) was administered at a dosage of 450 mg/day (100 mg active extract) in the absence of any other medication. The graphs of FIGS. 1a and 1b show the effect of Portulaca oleracea L. extract on non-insulin dependent diabetic patients with greater than and less than 300 mg/dl of blood glucose, respectively. It is evident from the graphs that Portulaca oleracea L. extract was capable of normalizing the glucose blood level in all patients of both groups, regardless of the initial blood glucose level. Note, blood glucose levels are considered diabetic according to the American Diabetes Association (ADA) criteria when a fasting blood glucose level is 126 mg/dl (7.0 mmol/L) or higher; a 2-hour oral glucose tolerance test result is 200 mg/dl (11.1 mmol/L) or higher.

Discussion

In both groups of Type 2 diabetes patients, a remarkable reduction in the level of blood glucose was observed. Diabetic patients whose blood glucose at the start of trial was less than 300 mg/dl maintained normal blood glucose levels after 2-3 weeks. Normal blood glucose levels were attained after 4-5 weeks in the group of diabetic patients with initial levels that were greater than 300 mg/dl.

### Example 3

### Examination of Toxic Effects in vitro of Portulaca oleracea L. Extracts of the Present Invention

The biosafety of Portulaca oleracea L. in vitro was investigated by a number of in vitro toxicology assays. The effects of Portulaca oleracea L. extracts on cell viability and cytotoxicity (MTT and LDH release assays), the expression of differentiated function in hepatocytes (albumin secretion assay), and on lipid peroxidation, a major indicator of oxidative stress (MDA assay) were examined. A human hepatocyte cell line, HepG2, co-cultures of hepatocytes and a monocyte cell line, THP1 (mimicking the physiological cellular environment), cells stimulated with lipopolysaccharides (LPS), a known activator of macrophages and hepatocytes, as well as tissue homogenate of sheep liver were incubated with various concentrations of Portulaca oleracea L. extracts and assayed. Standard procedure with measurements of toxicology. More than one type of cell is examined. Monocyte cells are white blood cells

Materials and Experimental Procedures

Plant extract—was generated as described in Example 2 above, only no binder was used. Briefly, 100 g of dried plant was extracted with 900 ml of ethanol-water mixture (80-20) for a period of 4 hours at 40° C. in a standard reflux set-up. The extract was filtered to remove plant residue, the ethanol was evaporated in vacuum at 50° C. and the water was removed by drying in a drying oven at 40° C. The extract (a sticky powdered material) was used as such and kept refrigerated when not in use.

Cell culture—The potential toxicity of the Portulaca oleracea L. extract was assessed in a cell culture system using the human hepatoplastoma cell line, HepG2 (ATCC ACCESSION NO. HB-8065), and the monocyte cell line, THP1 (ATCC ACCESSION NO. TIB-202). The HepG2 cell line retains differentiated parenchymal functions of normal hepatocytes, but can be grown indefinitely, permitting long-term studies. THP1 and HepG2 cells were grown in Dulbecco's modified Eagle's medium (DMEM) with a high glucose content (4.5 g/L) supplemented with 10% vol/vol inactivated fetal calf serum, 1% nonessential amino acids, 1% glutamine, 100 U/mL penicillin, and 10 μg/mL streptomycin. Cells were maintained in humidified atmosphere of 95% O₂-5% CO₂ at 37° C. The pH of the media was monitored to 7.4. Cell medium was changed twice a week. At 70-80% confluence, cells were trypsinized using 0.05% Trypsin and 0.02% EDTA for 5 min, centrifuged at 200 g for 10 min, resuspended in the culture medium and plated in microtiter dishes. 24 h following seeding, cells were exposed to various concentrations of the plant extracts in fresh serum-free medium.

MTT assay—2×10⁴ cells were seeded in 100 μl culture medium [i.e., Dulbecco's Modified Eagle's Medium (DMEM) with a high glucose content (4.5%) supplemented with 10% vol/vol inactivated calf serum, 1% nonessential amino acids, 1% glutamine 100 U/mll penicillin and 10 microg/mll streptomycin] in each well of 96-well microtiter dishes . 24 hrs following seeding, cells were incubated with various concentrations of water extracts of the plant for 24 hrs at 37° C. Following removal of the plant extracts from each well, cells were washed in phosphate buffered saline (PBS). The cells were then incubated in serum-free DMEM to which MTT (0.5 mg/ml) was added to each well (100 μl), and incubated for a further 4 hrs. To each well, 100 μL of an MTT solution was added (including 0.5 mg/ml MTT). The medium was then removed and the cells were incubated for 15 minutes (mins) with 100 μl of acidic isopropanol (0.08 N HCl) to dissolve the formazan crystals. The absorbance of MTT formazan was determined at 570 nm in an enzyme-linked immunosorbent assay (ELISA) reader. Viability was defined as the ratio, expressed as a percentage, of absorbance of treated cells to untreated cells.

Lactate dehydrogenase assay—2×10⁴ cells in 100 μl of DMEM medium (see above) were seeded in each well of a 96-well microtiter dish. Twenty four hrs following seeding, cells were exposed to increasing concentrations of the plant extracts (0.001-0.5 mg/ml). Following 24 hrs of incubation, the supernatants were carefully aspirated from each well. Cell monolayers were then treated with a cell lysis solution (100 μl) for 30 mins at room temperature and the resulting lysates were collected using micropipettes. LDH activity was measured in both the supernatants and the cell lysate fractions using the CytoTox 96 Nonradioactive Cytotoxicity Assay Kit (Promega, Wis., USA) in accordance with the manufacturer's instructions.

The LDH assay is based on the conversion of tetrazolium salt into a red formazan product, summarized by the following chemical reactions:

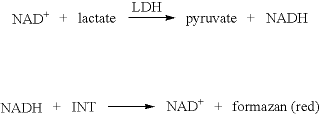

The intensity of the red color is proportional to LDH activity. Absorbance was determined at 490 nm in a 96-well plate ELISA reader. The percent of LDH released from the cells was determined using the formula:
LDH release=(Absorbance of the supernatant)/(absorbance of the supernatant+lysate)×100.

Albumin secretion assay—Quantification of albumin secreted from cells was effected as follows. 100 μl Culture supernatants were incubated in 96-well microtiter dishes for 1 hr at 37° C. or overnight at 4° C. Following a washing step, non-specific binding sites were blocked in PBS containing 0.5% bovine serum albumin (BSA) for 1 hr at RT. Following a second washing step, peroxidase-conjugated goat anti-rat albumin antibody was added in PBS containing 1% BSA and incubated for 2 hrs at RT. The microtiter dishes were then washed, and the substrate (0.5 mg/ml of 2.2-azino-di-3-ethylbenzothiazoline-6-sulfonic acid in 100 mM Na-acetate, 50 mM Na-phosphate, and 9×10⁻³% H₂O₂) was added. All washing steps were effected using PBS at RT. The absorption was measured at 405 nm in an ELISA reader. Background values were measured in the absence of culture supernatant and subtracted from the experimental values. All ELISA determinations were effected in duplicate.

MDA assay (6)—Measurement of lipid peroxidation using the MDA lipid peroxidation assay for thiobarbituric acid-reactive substances is based on the recovery of the MDA (malonic dialdehyde) made by adding a TBARS (thiobarbituric acid) reagent that gives a pink color when a reaction occurs between the reagent and the MDA (Diane W. Morel. Arteriosclerosis. Vol. 4, No. 4, 1984).

Preparation of TBARS reagents—The TBARS Solution (Working Reagent), containing 0.12 M TBA, pH=7.0, was prepared by adding water to a mixture of 0.375 g of TBA, 2.5 ml of concentrated HCl, and 15 ml of 100% TCA (tri-chloro acetic acid) to a final volume of 100 ml. The solution was heated to approximately 40° C. until the TBA dissolved.

An MDA standard solution containing 10 mM MDA was prepared as follows: 82 μl of MDA was added to 3.5 ml of concentrated HCl and brought to a final volume of 50 ml by addition of saline (0.9% NaCl). The MDA working standard solution containing 100 μM MDA was prepared by a 1:100 dilution of the MDA standard solution with saline.

Preparation of the MDA Standard Curve—Test tubes were prepared according to Table 12, below.

TABLE 12

| | Amount of MDA | Volume of Standard | Volume of DDW |
|---|---|---|---|
| | (nmol) | solution (μl) | (μl) |
| 1 | 0 | 0 | 500 |
| 2 | 0.75 | 7.5 | 492 |
| 3 | 1.50 | 15 | 485 |
| 4 | 4.5 | 45 | 455 |
| 5 | 7.5 | 75 | 425 |

DDW = double distilled water

To each tube, 1 ml of TBARS working reagent was added and the mixture was vortexed. Following incubation at 100° C. for 20 min, the mixture was centrifuged at 2000 rpm for 10 min and the O.D. of the supernatant was read at 522 nm versus a water blank.

MDA assay of plant extract—2 mg/ml of plant extract solution was prepared and then diluted according to Table 13 below, into new test tubes at the concentrations listed in column 1

TABLE 13

| 1 | 2 | 3 | | 5 |
|---|---|---|---|---|
| Concentration of | Volume of | Liver | 4 | Volume of |
| plant extract | plant extract | Homogenate | Volume of FeSO₄ | phosphate buffer |
| [mg/ml] | (μl) | (μl) | [10 mM] (μl) | pH = 7 (μl) |
| 0.00 | 0 | 200 | 10 | 790 |
| 0.01 | 5 | 200 | 10 | 785 |
| 0.05 | 25 | 200 | 10 | 765 |
| 0.10 | 50 | 200 | 10 | 740 |
| 0.25 | 125 | 200 | 10 | 665 |
| 0.50 | 250 | 200 | 10 | 540 |

The test tubes were incubated in a water bath at 37° C. for 30 min with occasional shaking. Following centrifugation at 3500 rpm (2900×g) for 10 min, 0.5 ml of the supernatant was transferred to a new test tube. Following addition of 1 ml of TBARS working solution, the mixture was vortexed, heated at 100° C. for 20 min, and then centrifuged at 2000 rpm for 10 min. The O.D. of the supernatant was read at 522 nm versus a water blank. The amount of MDA was calculated from the standard curve according to the following equation:

$$nmol MDA / mg protein = nmol MDA * \frac{1000}{volumeofsample ( µl} * \frac{1}{( mg ) protein / ml}$$

Results

Portulaca oleracea L. extracts do not affect in vitro cell growth as determined by the MTT assay—The tetrazolium dye, MTT, is widely used to assess the viability and/or the metabolic state of cells. This calorimetric assay is based on the conversion of the yellow tetrazolium bromide (MTT) to the red formazan derivative by mitochondrial succinate dehydrogenase in viable cells.

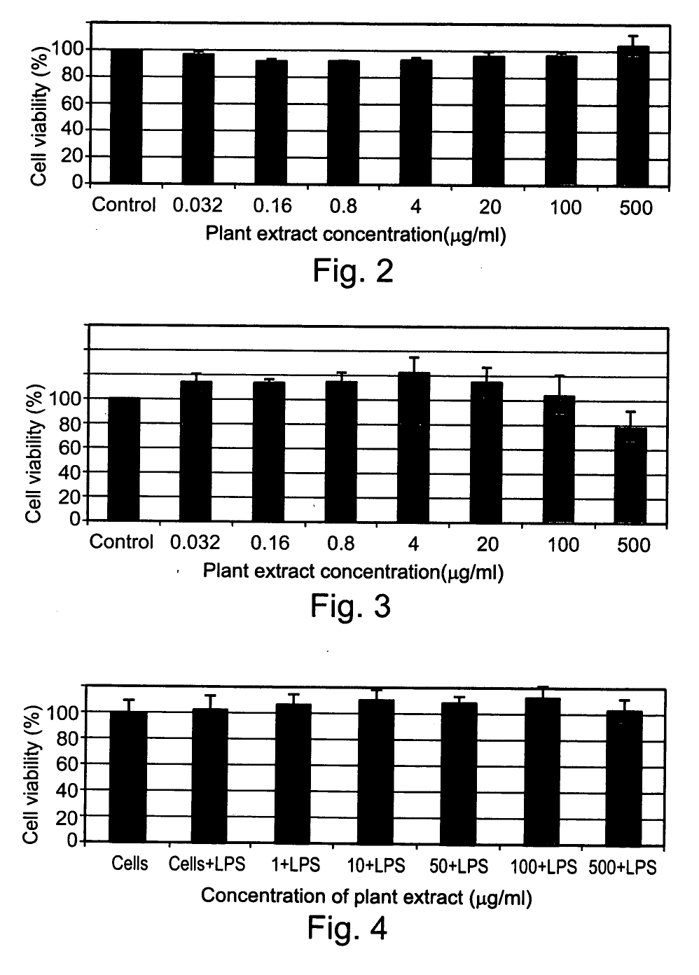

HepG2—To evaluate the biosafety of the ethanol-water Portulaca oleracea L. extract, HepG2 cells were incubated with increasing concentrations of the Portulaca oleracea L. extract for 24 hrs. Following removal of the plant extracts from each well, cells were washed in PBS, and the MTT assay was effected, as described in Example 3, Materials and Experimental Procedures. As observed in FIG. 2, extract from Portulaca oleracea L. exhibited no obvious negative effects at any of the concentrations tested.

Co-cultures of HepG2 and THP1—To further evaluate the biosafety of the ethanol-water Portulaca oleracea L. extract, the effect of increasing concentrations of the extract on co-cultures of HepG2 cells and the monocyte cell line, THP1, following a 24 hr incubation was examined using the MTT assay, effected as described above. As observed in FIG. 3, extracts from Portulaca oleracea L. exhibited no sign of any negative effects at all concentrations tested. Up to 100 μg/ml (the highest concentration tested, the extract showed no effect on cell viability.

Lipopolysacchride (LPS)-treated co-cultures of HepG2 and THP1—To evaluate the effects of plant extract on LPS-activated cells, co-cultures of HepG2 cells (2×10⁴), and THP1 cells (5×10³) were seeded in 100 μl of medium in each well of 96-well dishes. Twenty four hrs following seeding, cells were incubated with increasing concentrations of the ethanol-water Portulaca oleracea L. extract in the absence and the presence of 10 μg LPS/μl for 24 hrs at 37° C. Following careful aspiration of the supernatant from each well, cells were washed in PBS and the MTT assay was effected, as described in Example 3, Materials and Experimental Procedures. FIG. 4 summarizes the results of the MTT assay in HepG2 and THP1 co-cultures following an overnight co-incubation with LPS and increasing concentrations of the Portulaca oleracea L. extract. The MTT test showed no decrease in veil viability up to the maximum level tested, i.e. 500 μgr/mll.

Portulaca oleracea L. extracts do not affect membrane integrity—Membrane integrity may be evaluated by measurement of lactate dehydrogenase activity, LDH, which catalyses the conversion of lactate to pyruvate. In the LDH assay, leakage of the exclusively cytosolic enzyme, LDH, into the extracellular medium is measured. When cells are disrupted, the LDH activity is elevated. The presence of LDH in the cell culture medium is indicative of damage to the cell membrane either due to cellular death or a leak in a cell membrane.

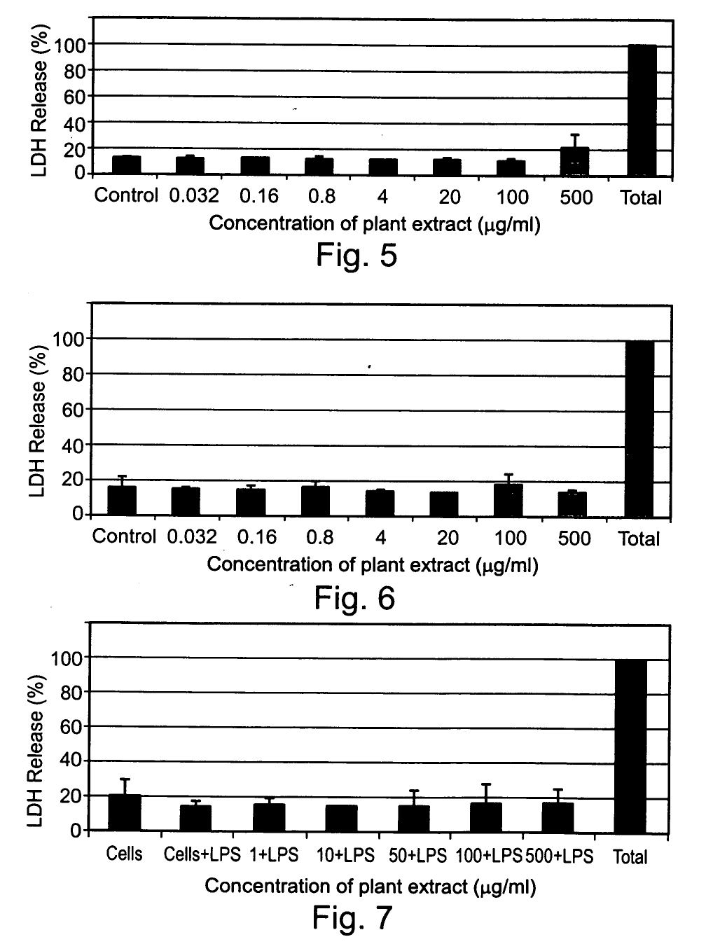

HepG2 cells—HepG2 cells were incubated with increasing concentrations of Portulaca oleracea L. extracts for 24 hrs and the LDH assay was effected, as described in Example 3, Materials and Experimental Procedures. FIG. 5 summarizes LDH leakage as percentage of total (i.e., total amount of LDH after cell destruction) from HepG2 cells following an overnight incubation with increasing concentrations of the ethanol-water Portulaca oleracea L. extract. FIG. 6 summarizes the percentage of LDH leakage from a HepG2-THP1 cell co-culture following an overnight incubation with various concentrations of the ethanol-water Portulaca oleracea L. extracts. Thus, a 24 hr incubation of Portulaca oleracea L. extract with HepG2-THP1 cells induced no significant change in the LDH levels in the culture medium.

LPS-treated co-cultures of HepG2 and THP1—FIG. 7 summarizes the percentage of LDH leakage from HepG2 and THP1 co-cultured cells following an overnight co-incubation with 10 μg/ml of LPS and increasing concentrations of the Portulaca oleracea L. extracts. As observed with HepG2 cells alone, LDH levels in the medium of LPS-treated HepG2 and THP1 co-cultures were not significantly increased following incubation with ethanol-water Portulaca oleracea L. extract.

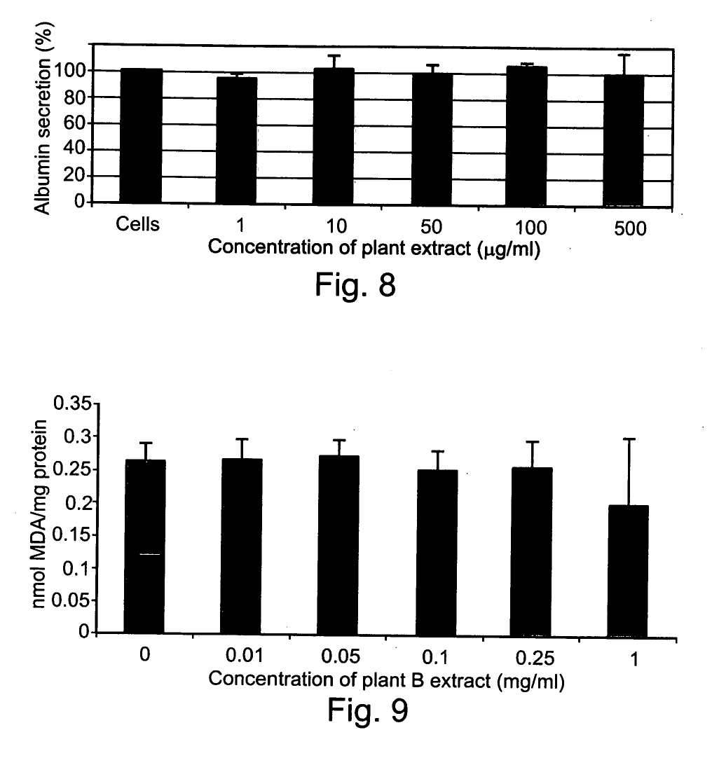

Portulaca oleracea L. extracts do not affect cell function—The effect of Portulaca oleracea L. extract on the cell type-specific expression of proteins was investigated as a means to examine differentiated function. Expression of liver-specific function was assessed by measurement of the secretion of albumin by HepG2 cells using the albumin secretion assay, as described in Example 3, Materials and Experimental Procedures. FIG. 8 displays albumin production in HepG2 cells following an overnight incubation with increasing concentrations of the Portulaca oleracea L. extracts. The levels of albumin in the cell culture medium were found to be unchanged by incubation with the Portulaca oleracea L. extract.

Portulaca oleracea L. extracts do not induce oxidative stress—The sensitivity of measuring Thiobarbituric Acid-Reactive Substances (TBARS) has made the MDA assay the method of choice for screening and monitoring lipid peroxidation, a major indicator of oxidative stress (1-3). This rapid, easy-to-use procedure has been modified by researchers for use with many types of samples including drugs, food products, as well as human and animal biological tissues (4-7). The MDA assay has provided important information regarding free radical activity in disease states and has been used for measurement of anti-oxidant activity of several compounds.

MDA release was measured as a function of the concentration of Portulaca oleracea L. extract in sheep liver homogenate. FIG. 9 displays the results of increasing concentrations of Portulaca oleracea L. alcohol-water extract on 10 μM FeSO₄-induced lipid peroxidation in sheep liver homogenate. No change in free radical concentration was observed, indicating that the extract has no effect on peroxidation processes.

Discussion

The MTT, LDH release, albumin secretion, and MDA assays indicated that ethanol-water extract of Portulaca oleracea L. is biosafe as it caused no toxic effects or any effect on differentiated function in all parameters measured nor any effect on the concentration of free radicals up to extract concentrations of 1000 μg/ml.

### Example 4

### Examination of the Efficacy in vitro of Portulaca oleracea L. Extracts

The preliminary experiments of Example 2 above demonstrated that Portulaca oleracea L. extract reduced the level of glucose in the bloodstream of diabetic patients. The complexity of diabetes type 2 makes it impossible to develop a single in vitro assay that would duplicate the efficacy observed in human subjects (where the reduction in glucose levels is an overall effect without indication as to how it is achieved). Thus, it is of importance to mimic as closely as possible the effects of the Portulaca oleracea L. extract on transport as well as on adsorption of glucose. Several standard assays that measure the transport of glucose from the medium (blood) through the cell membrane into the cell are available. In addition, in vitro measurements of the adsorption of glucose through the walls of the small intestine into the bloodstream, providing an indication of the first stage of glucose metabolism, may be modeled using adsorption through animal intestinal tissue. Two assays were effected to determine the effect of Portulaca oleracea L. extract on glucose adsorption and transport.

### Example 4a Describes the Effect of a Portulaca oleracea L. Extract on Glucose Adsorption through the Intestine

Materials and Experimental Procedures

Measurement of the effect of Portulaca oleracea L. extract on glucose adsorption through the intestine—A 10 cm length of sheep small intestine was connected to a container, which held a Krebs solution containing 100 mg/ml of D-glucose and a relevant amount of Portulaca oleracea L. extract, on one side and to a collecting container on the other side, such that the Krebs solution flowed through the intestine. The flow rate of the solution into the system was 50-55 ml/hr. The intestine was incubated in a thermostrated bath containing DDW at 37° C. Sugar is adsorbed through the intestine and transferred through the walls to the water bath. Samples from the bath were taken every 15 mins and the glucose concentration of the samples was tested according to the DNS method, which is a general method for measuring glucose concentration, described herein below.

Preparation of the DNS Reagent—7.49 g of 3,5-dinitrosalicylic acid (DNS) and 14 g of NaOH were added to 1 l of DDW and, mixed. When dissolved, 216 g of Rochelle salts (Na-K-tartrate), 5.37 g of phenol, and 5.86 g of sodium meta bisulfate were then added and mixed.

DNS method—Three ml of DNS Reagent were added to 1 ml of sample and the mixture was incubated in boiling water (100° C.) for 5 mins and then transferred to ice until it reached room temperature. The O.D. was measured at 640 nm using a spectrophotometer and results were calculated from a standard curve of dextrose at concentrations of: 0, 0.2, 0.4, 0.6, 0.8, and 1.0 mg/ml.

Results

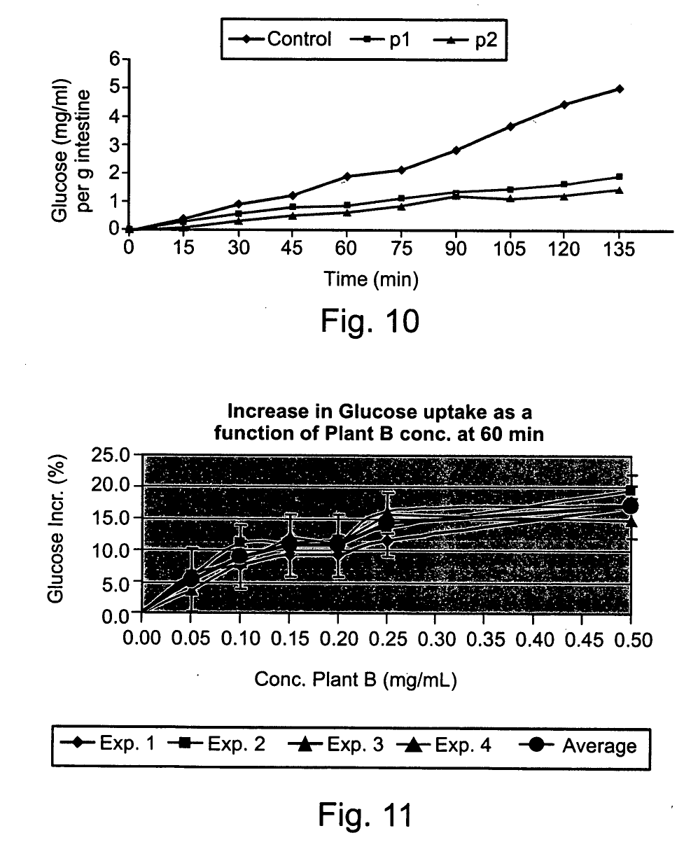

FIG. 10 displays the effect of Portulaca oleracea L. extract on glucose adsorption through the sheep intestine. Measurements were normalized for the weight of the intestine sample, which is indicative of its protein content. It is evident from the graph that the Portulaca oleracea L. extract partially inhibited the transport of glucose through the intestine, indicating that a reduction of glucose levels in the bloodstream would occur.

### Example 4b Describes the Effect of Portulaca oleracea L. on the Transport of Glucose into Yeast Cells

Measurement of the transport of glucose into yeast cells, a method that has been in use for many years, exploits the evolution of CO₂ by yeast cells as an indicator of activity, which is a direct response to glucose concentration.

Materials and Experimental Procedures

Glucose transport assay—2.5 ml of a stock 40 mg/ml solution of bakers yeast (Saccharomyces cerevisiae) and increasing amounts (from 0.05 mg/ml to 0.5 mg/ml) of a stock solution of Portulaca oleracea L. extract (1 mg/ml of standard ethanol-water (80:20) extract whose volume was adjusted to a total of 24 ml) were added to a 100 ml serum bottle, which was maintained under nitrogen and connected to a graduated manometer to measure gas evolution. The experiment was also effected with a blank consisting of yeast solution without Portulaca oleracea L. extract.

The bottles were shaken in a thermostrated shaker bath at 37° C. CO₂ evolution commences immediately but ceases after a short period when the system reaches equilibrium. At that point, 1 ml of 0.15 g/ml glucose was added and the amount of glucose evolved was measured and recorded as a function of time, every 5 mins over a period of 1 hr. After 1 hr, the experiment was halted. The amount of CO₂ recorded was converted into mg of glucose based upon a conversion of 1 g CO₂=2.04 g glucose. The amounts of glucose consumed in the presence and absence of Portulaca oleracea L. extract was depicted over time in a graph .

Results

A measured number of yeast cells was maintained under controlled conditions and fed specific amounts of glucose, and the evolution of CO₂ was measured. The experiment was effected in duplicate, whereby Portulaca oleracea L. extract was added in addition to glucose in the second experiment. The change in the amount of CO₂ evolved is a measure of the effect of Portulaca oleracea L. extract on glucose transport into the cell.

It is evident from FIG. 11, which summarizes the effect of Portulaca oleracea L. extract on glucose uptake in yeast cells, that Portulaca oleracea L. extract increased the CO₂ output of the yeast cells. This indicates increased activity due to an increase in transport of glucose into the cell caused by Portulaca oleracea L. extract.

Discussion

The above-described in vitro experiments, as well as preliminary trials on human subjects with diabetes, have demonstrated Portulaca oleracea L. extract to be both biosafe and effective in the reduction of glucose levels in the bloodstream.

In vitro experiments have shown that the extract influences transport of glucose into the cell, as well as the adsorption of glucose through the small intestine. This may indicate the presence of at least two active ingredients. Purification of the extract and isolation of the active components will allow a more in-depth study of the mechanism of the extract.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES CITED BY NUMERALS

### Other References are Cited in Their Entirety throughout the Application

1. USDA, NRCS. 2002. The PLANTS Database, Version 3.5 (plants.usda.gov). National Plant Data Center, Baton Rouge, La. 70874-4490 USA.

2. USDA, ARS, National Genetic Resources Program. Phytochemical and Ethnobotanical Databases. [Online Database] National Germiplasm Resources Laboratory, Beltsville, Md. Description of P.O.

3. USDA, ARS, National Genetic Resources Program. Phytochemical and Ethnobotanical Databases. [Online Database] National Germplasm Resources Laboratory, Beltsville, Md. 30 Jul. 2003 Activities of P.O.

4. Hypoglycaemic and Hyperinsulinemic effects of some Egyptian Herbs used for the treatment of Diabetes Mellitus (type II) in rats; E. F. Eskander and H. Won Jun; Egypt. J. Pharm. Sci. No.1-6, 331-342 (1995)

5. In Vitro Evaluation of Biosafety of Plant Extracts from D-Herb; SUHA DAKWAR, BASHAR SAAD; The R&D Center of the Galilee Society, Shfar-Am, 2003

6. In Vitro Evaluation of Anti-Oxidative Stress of Portulaca oleracea L. Extract; Khaled Abu Saleh, Sobhi Sauob; D-Herb Ltd. Internal Report May 2003

1-102. (canceled)

103. A method of isolating anti hyperglycemic agents from Portulaca oleracea L., the method comprising
(a) extracting polar components from Portulaca oleracea L.; and
(b) purifying said polar components thereby isolating the anti hyperglycemic agents from Portulacea oleracea L.

104. The method of claim 103, wherein said extracting is effected by employing a solvent gradient of increasing polarity

105. The method of claim 103, wherein said extracting is effected by ethanol-water extraction.

106. The method of claim 104, wherein said solvents of increasing polarity are hexane, ethyl acetate, dichloromethane, methanol and water.

107. The method of claim 104, wherein said solvents of increasing polarity are hexane:dichloromethane:ethylacetate (1:1:1) and methanol:ethanol:water (1:1:1).

108. The method of claim 103, wherein said purifying said polar components from said extract is effected by thin layer chromatography.

109. A method of isolating anti hyperglycemic agents from Portulaca oleracea L., the method comprising extracting non-polar components from Portulaca oleracea L., thereby isolating the anti hyperglycemic agents from Portulaca oleracea L.

110. The method of claim 109, wherein said extracting is effected using non-polar solvents.

111. The method of claim 109, wherein said extracting is effected by ethanol-water extraction.

112. The method of claim 110, wherein said non-polar solvents are selected from the group consisting of hexane, dichloromethane and ethyl acetate.

113. The method of claim 109, further comprising purifying said non-polar components from said extract.

114. The method of claim 113, wherein said purifying said non-polar components from said extract is effected by thin layer chromatography.

115. A composition of matter comprising an ethanol-water extract of Portulacea oleracea L.

116. The composition of matter of claim 115, wherein a ratio of said ethanol-water is 80% -20%.

117. The composition of matter of claim 115, wherein said ethanol-water extract of Portulaca oleracea L. is capable of lowering glucose levels in the blood.

118. The composition-of-matter of claim 117, wherein said ethanol-water extract of Portulaca oleracea L. is capable of increasing glucose transport into cells and/or decreasing glucose adsorption through the intestines.

119. A composition of matter comprising a polar fraction extract of Portulacea oleracea L.

120. The composition of matter wherein said polar fraction extract does not comprise hydrocolloids.

121. The composition-of-matter of claim 119, wherein said polar extract is capable of lowering glucose levels in the blood.

122. The composition-of-matter of claim 119, wherein said polar extract is capable of increasing glucose transport into cells.

123. The composition-of-matter of claim 119, wherein said composition has Rf values in a range of 0.0-0.45 when subjected to thin-layer chromatographic fractionation on Silica Gel 60 F254 on aluminum using a solvent of dichloromethane:hexane:methanol in proportions of 1:1:0.2.

124. The composition-of-matter of claim 119, wherein said composition has Rf values in a range of 0.0-0.32 when subjected to thin-layer chromatographic fractionation on Silica Gel 60 F254 on aluminum using a solvent of dichloromethane:hexane:methanol in proportions of 1:1:0.2.

125. The composition-of-matter of claim 119, wherein said composition has Rf values in a range of 0.17-0.41 when subjected to thin-layer chromatographic fractionation on Silica Gel 60 F254 on aluminum using a solvent of dichloromethane:hexane:methanol in proportions of 1:1:0.2.

126. The composition-of matter of claim 119, wherein said polar fraction extract of Portulaca oleracea L. is extracted by Soxlett extraction using methanol and has a Rf value selected from the group consisting of 0.0, 0.31, 0.34, 0.36, 0.39 and 0.45 when subjected to thin-layer chromatographic fractionation on Silica Gel 60 F254 on aluminum using a solvent of dichloromethane:hexane:methanol in proportions of 1:1:0.2.

127. The composition-of matter of claim 119, wherein said polar fraction extract of Portulaca oleracea L. is extracted by Soxlett extraction using water and has a Rf value of 0.0 when subjected to thin-layer chromatographic fractionation on Silica Gel 60 F254 on aluminum using a solvent of dichloromethane:hexane:methanol in proportions of 1:1:0.2.

128. The composition-of matter of claim 119, wherein said polar fraction extract of Portulaca oleracea L. is extracted with methanol and has a Rf value selected from the group consisting of 0.0, 0.15, 0.30 and 0.32 when subjected to thin-layer chromatographic fractionation on Silica Gel 60 F254 on aluminum using a solvent of dichloromethane:hexane:methanol in proportions of 1:1:0.2.

129. The composition-of matter of claim 119, wherein said polar fraction extract of Portulaca oleracea L. is extracted with methanol:ethanol:water in proportions of 1:1:1 and has a Rf value selected from the group consisting of 0.17, 0.27, 0.30, 0.34, 0.39 and 0.41 when subjected to thin-layer chromatographic fractionation on Silica Gel 60 F254 on aluminum using a solvent of dichloromethane:hexane:methanol in proportions of 1:1:0.2.

130. A composition-of-matter comprising a non-polar fraction extract of Portulacea oleracea L.

131. The composition-of-matter of claim 130, wherein said non-polar extract is capable of decreasing glucose levels in the blood.

132. The composition-of-matter of claim 130, wherein said non-polar extract is capable of decreasing glucose adsorption through the intestines.

133. The composition-of-matter of claim 130, wherein said composition has Rf values in a range of 0.11-0.89 when subjected to thin-layer chromatographic fractionation on Silica Gel 60 F254 on aluminum using a solvent of dichloromethane:hexane:methanol in proportions of 1:1:0.2.

134. The composition-of-matter of claim 130, wherein said composition has Rf values in a range of 0.11-0.88 when subjected to thin-layer chromatographic fractionation on Silica Gel 60 F254 on aluminum using a solvent of dichloromethane:hexane:methanol in proportions of 1:1:0.2.

135. The composition-of-matter of claim 130, wherein said composition has Rf values in a range of 0.17-0.91 when subjected to thin-layer chromatographic fractionation on Silica Gel 60 F254 on aluminum using a solvent of dichloromethane:hexane:methanol in proportions of 1:1:0.2.

136. The composition-of matter of claim 130, wherein said non-polar fraction extract of Portulaca oleracea L. is extracted by Soxlett extraction using hexane and has a Rf value selected from the group consisting of 0.36, 0.45, 0.52, 0.71 and 0.88 when subjected to thin-layer chromatographic fractionation on Silica Gel 60 F254 on aluminum using a solvent of dichloromethane:hexane:methanol in proportions of 1:1:0.2.

137. The composition-of matter of claim 130, wherein said non-polar fraction extract of Portulaca oleracea L. is extracted by Soxlett extraction using ethyl acetate and has a Rf value selected from the group consisting of 0.11, 0.18, 0.31, 0.36, 0.45, 0.52 and 0.71 when subjected to thin-layer chromatographic fractionation on Silica Gel 60 F254 on aluminum using a solvent of dichloromethane:hexane:methanol in proportions of 1:1:0.2.

138. The composition-of matter of claim 130, wherein said non-polar fraction extract of Portulaca oleracea L. is extracted with hexane and has a Rf value selected from the group consisting of 0.3, 0.32, 0.41, 0.47 and 0.89 when subjected to thin-layer chromatographic fractionation on Silica Gel 60 F254 on aluminum using a solvent of dichloromethane:hexane:methanol in proportions of 1:1:0.2.

139. The composition-of matter of claim 130, wherein said non-polar fraction extract of Portulaca oleracea L. is extracted with ethyl acetate and has a Rf value selected from the group consisting of 0.15, 0.36, 0.47, 0.73 and 0.89 when subjected to thin-layer chromatographic fractionation on Silica Gel 60 F254 on aluminum using a solvent of dichloromethane:hexane:methanol in proportions of 1:1:0.2.

140. The composition-of matter of claim 130, wherein said non-polar fraction extract of Portulaca oleracea L. is extracted with hexane:DCM:ethyl-acetate in proportions of 1:1:1 and has a Rf value selected from the group consisting of 0.17, 0.30, 0.36, 0.41, 0.68 and 0.91 when subjected to thin-layer chromatographic fractionation on Silica Gel 60 F254 on aluminum using a solvent of dichloromethane:hexane:methanol in proportions of 1:1:0.2.

141. A pharmaceutical composition for reducing blood glucose levels comprising a therapeutic effective amount of a composition including a polar fraction extract of Portulaca oleracea L. and a pharmaceutical acceptable carrier or diluent.

142. The pharmaceutical composition of claim 141, wherein said polar fraction extract has Rf values in a range of 0.0-0.45 when subjected to thin-layer chromatographic fractionation on Silica Gel 60 F254 on aluminum using a solvent of dichloromethane:hexane:methanol in proportions of 1:1:0.2.

143. The pharmaceutical composition of claim 141, wherein said polar fraction extract has Rf values in a range of 0.0-0.32 when subjected to thin-layer chromatographic fractionation on Silica Gel 60 F254 on aluminum using a solvent of dichloromethane:hexane:methanol in proportions of 1:1:0.2.

144. The pharmaceutical composition of claim 141, wherein said polar fraction extract has Rf values in a range of 0.17-0.41 when subjected to thin-layer chromatographic fractionation on Silica Gel 60 F254 on aluminum using a solvent of dichloromethane:hexane:methanol in proportions of 1:1:0.2.

145. The pharmaceutical composition of claim 141, wherein said polar fraction extract of Portulaca oleracea L. is extracted by Soxlett extraction using methanol and has a Rf value selected from the group consisting of 0.0, 0.31, 0.34, 0.36, 0.39 and 0.45 when subjected to thin-layer chromatographic fractionation on Silica Gel 60 F254 on aluminum using a solvent of dichloromethane:hexane:methanol in proportions of 1:1:0.2.

146. The pharmaceutical composition of claim 141, wherein said polar fraction extract of Portulaca oleracea L. is extracted by Soxlett extraction using water and has a Rf value of 0.0 when subjected to thin-layer chromatographic fractionation on Silica Gel 60 F254 on aluminum using a solvent of dichloromethane:hexane:methanol in proportions of 1:1:0.2.

147. The pharmaceutical composition of claim 141, wherein said polar fraction extract of Portulaca oleracea L. is extracted with methanol and has a Rf value selected from the group consisting of 0.0, 0.15, 0.30 and 0.32 when subjected to thin-layer chromatographic fractionation on Silica Gel 60 F254 on aluminum using a solvent of dichloromethane:hexane:methanol in proportions of 1:1:0.2.

148. The pharmaceutical composition of claim 141, wherein said polar fraction extract of Portulaca oleracea L. is extracted with methanol:ethanol:water in proportions of 1:1:1 and has a Rf value selected from the group consisting of 0.17, 0.27, 0.30, 0.34, 0.39 and 0.41 when subjected to thin-layer chromatographic fractionation on Silica Gel 60 F254 on aluminum using a solvent of dichloromethane:hexane:methanol in proportions of 1:1:0.2.

149. The pharmaceutical composition of claim 141, wherein said polar fraction extract does not comprise hydrocolloids.

150. A pharmaceutical composition for decreasing blood glucose levels comprising a therapeutic effective amount of a composition including a non-polar fraction extract of Portulaca oleracea L and a pharmaceutical acceptable carrier or diluent.

151. The pharmaceutical composition of claim 150, wherein said non-polar fraction extract has Rf values in a range of 0.11-0.89 when subjected to thin-layer chromatographic fractionation on Silica Gel 60 F254 on aluminum using a solvent of dichloromethane:hexane:methanol in proportions of 1:1:0.2.

152. The pharmaceutical composition of claim 150, wherein said non-polar fraction extract has Rf values in a range of 0.11-0.88 when subjected to thin-layer chromatographic fractionation on Silica Gel 60 F254 on aluminum using a solvent of dichloromethane:hexane:methanol in proportions of 1:1:0.2.

153. The pharmaceutical composition of claim 150, wherein said non-polar fraction extract has Rf values in a range of 0.17-0.91 when subjected to thin-layer chromatographic fractionation on Silica Gel 60 F254 on aluminum using a solvent of dichloromethane:hexane:methanol in proportions of 1:1:0.2.

154. The pharmaceutical composition of claim 150, wherein said non-polar fraction extract of Portulaca oleracea L. is extracted by Soxlett extraction using hexane and has a Rf value selected from the group consisting of 0.36, 0.45, 0.52, 0.71 and 0.88 when subjected to thin-layer chromatographic fractionation on Silica Gel 60 F254 on aluminum using a solvent of dichloromethane:hexane:methanol in proportions of 1:1:0.2.

155. The pharmaceutical composition of claim 150, wherein said non-polar fraction extract of Portulaca oleracea L. is extracted by Soxlett extraction using ethyl acetate and has a Rf value selected from the group consisting of 0.11, 0.18, 0.31, 0.36, 0.45, 0.52 and 0.71 when subjected to thin-layer chromatographic fractionation on Silica Gel 60 F254 on aluminum using a solvent of dichloromethane:hexane:methanol in proportions of 1:1:0.2.

156. The pharmaceutical composition of claim 150, wherein said non-polar fraction extract of Portulaca oleracea L. is extracted with hexane and has a Rf value selected from the group consisting of 0.3, 0.32, 0.41, 0.47 and 0.89 when subjected to thin-layer chromatographic fractionation on Silica Gel 60 F254 on aluminum using a solvent of dichloromethane:hexane:methanol in proportions of 1:1:0.2.

157. The pharmaceutical composition of claim 150, wherein said non-polar fraction extract of Portulaca oleracea L. is extracted with ethyl acetate and has a Rf value selected from the group consisting of 0.15, 0.36, 0.47, 0.73 and 0.89 when subjected to thin-layer chromatographic fractionation on Silica Gel 60 F254 on aluminum using a solvent of dichloromethane:hexane:methanol in proportions of 1:1:0.2.

158. The pharmaceutical composition of claim 150, wherein said non-polar fraction extract of Portulaca oleracea L. is extracted with hexane:DCM:ethyl-acetate in proportions of 1:1:1 and has a Rf value selected from the group consisting of 0.17, 0.30, 0.36, 0.41, 0.68 and 0.91 when subjected to thin-layer chromatographic fractionation on Silica Gel 60 F254 on aluminum using a solvent of dichloromethane:hexane:methanol in proportions of 1:1:0.2.

159. A method of treating a hyperglycemia-related disease in a subject, the method comprising administering to a subject in need thereof a therapeutic effective amount of a composition including an ethanol-water extract of Portulaca oleracea L., thereby treating the hyperglycemia-related disease in the subject.

160. A method of treating a hyperglycemia-related disease in a subject, the method comprising administering to a subject in need thereof a therapeutic effective amount of a composition including a polar fraction extract of Portulaca oleracea L., thereby treating the hyperglycemia-related disease in the subject.

161. The method of claim 160, wherein said polar fraction does not comprise hydrocolloids.

162. The method of claim 160, wherein the hyperglycemia-related disease is selected from the group consisting of diabetes, Cushing's disease, Cushing's syndrome, eating disorders, impaired glucose tolerance, glomerular microangiopathy, diffuse glomerulosclerosis, nodular glomerulosclerosis, urinary infections, acute pyelonephritis, necrotizing papillitis, emphysematous pyelonephritis, glycogen nephrosis (armanni-ebstein lesion), retinopathy, nonproliferative retinopathy, capillary microaneurysms, retinal edema exudates, hemorrhages, proliferative retinopathy, proliferation of small vessels, hemorrhage fibrosis, retinal detachment, cataracts, transient refractive errors due to osmotic changes in lens, glaucoma due to proliferation of vessels in the iris, retinal infections, cerebrovascular atherosclerotic disease, neuropathy, skin infections, coronary atherosclerosis, myocardial infarction, peripheral atherosclerosis: limb ischemia, gangrene, increased fetal death rate, increased susceptibility to infection and delayed wound healing.

163. The method of claim 160, wherein said polar extract is capable of lowering glucose levels in the blood.

164. The method of claim 160, wherein said polar fraction extract has Rf values in a range of 0.0-0.45 when subjected to thin-layer chromatographic fractionation on Silica Gel 60 F254 on aluminum using a solvent of dichloromethane:hexane:methanol in proportions of 1:1:0.2.

165. The method of claim 160, wherein said polar fraction extract has Rf values in a range of 0.0-0.32 when subjected to thin-layer chromatographic fractionation on Silica Gel 60 F254 on aluminum using a solvent of dichloromethane:hexane:methanol in proportions of 1:1:0.2.

166. The method of claim 160, wherein said polar fraction extract has Rf values in a range of 0.17-0.41 when subjected to thin-layer chromatographic fractionation on Silica Gel 60 F254 on aluminum using a solvent of dichloromethane:hexane:methanol in proportions of 1:1:0.2.

167. The method of claim 160, wherein said polar fraction extract of Portulaca oleracea L. is extracted by Soxlett extraction using methanol and has a Rf value selected from the group consisting of 0.0, 0.31, 0.34, 0.36, 0.39 and 0.45 when subjected to thin-layer chromatographic fractionation on Silica Gel 60 F254 on aluminum using a solvent of dichloromethane:hexane:methanol in proportions of 1:1:0.2.

168. The method of claim 160, wherein said polar fraction extract of Portulaca oleracea L. is extracted by Soxlett extraction using water and has a Rf value of 0.0 when subjected to thin-layer chromatographic fractionation on Silica Gel 60 F254 on aluminum using a solvent of dichloromethane:hexane:methanol in proportions of 1:1:0.2.

169. The method of claim 160, wherein said polar fraction extract of Portulaca oleracea L. is extracted with methanol and has a Rf value selected from the group consisting of 0.0, 0.15, 0.30 and 0.32 when subjected to thin-layer chromatographic fractionation on Silica Gel 60 F254 on aluminum using a solvent of dichloromethane:hexane:methanol in proportions of 1:1:0.2.

170. The method of claim 160, wherein said polar fraction extract of Portulaca oleracea L. is extracted with methanol:ethanol:water in proportions of 1:1:1 and has a Rf value selected from the group consisting of 0.17, 0.27, 0.30, 0.34, 0.39 and 0.41 when subjected to thin-layer chromatographic fractionation on Silica Gel 60 F254 on aluminum using a solvent of dichloromethane:hexane:methanol in proportions of 1:1:0.2.

171. A method of treating a hyperglycemia-related disease in a subject, the method comprising administering to a subject in need thereof a therapeutic effective amount of a composition including a non-polar fraction extract of Portulaca oleracea L., thereby treating the hyperglycemia-related disease in the subject.

172. The method of claim 171, wherein the hyperglycemia-related disease is selected from the group consisting of diabetes, Cushing's disease, Cushing's syndrome, eating disorders, impaired glucose tolerance, glomerular microangiopathy, diffuse glomerulosclerosis, nodular glomerulosclerosis, urinary infections, acute pyelonephritis, necrotizing papillitis, emphysematous pyelonephritis, glycogen nephrosis (armanni-ebstein lesion), retinopathy, nonproliferative retinopathy, capillary microaneurysms, retinal edema exudates, hemorrhages, proliferative retinopathy, proliferation of small vessels, hemorrhage fibrosis, retinal detachment, cataracts, transient refractive errors due to osmotic changes in lens, glaucoma due to proliferation of vessels in the iris, retinal infections, cerebrovascular atherosclerotic disease, neuropathy, skin infections, coronary atherosclerosis, myocardial infarction, peripheral atherosclerosis: limb ischemia, gangrene, increased fetal death rate, increased susceptibility to infection and delayed wound healing.

173. The method of claim 171, wherein said non-polar fraction extract has Rf values in a range of 0.11-0.89 when subjected to thin-layer chromatographic fractionation on Silica Gel 60 F254 on aluminum using a solvent of dichloromethane:hexane:methanol in proportions of 1:1:0.2.

174. The method of claim 171, wherein said non-polar fraction extract has Rf values in a range of 0.11-0.88 when subjected to thin-layer chromatographic fractionation on Silica Gel 60 F254 on aluminum using a solvent of dichloromethane:hexane:methanol in proportions of 1:1:0.2.

175. The method of claim 171, wherein said non-polar fraction extract has Rf values in a range of 0.17-0.91 when subjected to thin-layer chromatographic fractionation on Silica Gel 60 F254 on aluminum using a solvent of dichloromethane:hexane:methanol in proportions of 1:1:0.2.

176. The method of claim 171, wherein said non-polar fraction extract of Portulaca oleracea L. is extracted by Soxlett extraction using hexane and has a Rf value selected from the group consisting of 0.36, 0.45, 0.52, 0.71 and 0.88 when subjected to thin-layer chromatographic fractionation on Silica Gel 60 F254 on aluminum using a solvent of dichloromethane:hexane:methanol in proportions of 1:1:0.2.

177. The method of claim 171, wherein said non-polar fraction extract of Portulaca oleracea L. is extracted by Soxlett extraction using ethyl acetate and has a Rf value selected from the group consisting of 0.11, 0.18, 0.31, 0.36, 0.45, 0.52 and 0.71 when subjected to thin-layer chromatographic fractionation on Silica Gel 60 F254 on aluminum using a solvent of dichloromethane:hexane:methanol in proportions of 1:1:0.2.

178. The method of claim 171, wherein said non-polar fraction extract of Portulaca oleracea L. is extracted with hexane and has a Rf value selected from the group consisting of 0.3, 0.32, 0.41, 0.47 and 0.89 when subjected to thin-layer chromatographic fractionation on Silica Gel 60 F254 on aluminum using a solvent of dichloromethane:hexane:methanol in proportions of 1:1:0.2.

179. The method of claim 171, wherein said non-polar fraction extract of Portulaca oleracea L. is extracted with ethyl acetate and has a Rf value selected from the group consisting of 0.15, 0.36, 0.47, 0.73 and 0.89 when subjected to thin-layer chromatographic fractionation on Silica Gel 60 F254 on aluminum using a solvent of dichloromethane:hexane:methanol in proportions of 1:1:0.2.

180. The method of claim 171, wherein said non-polar fraction extract of Portulaca oleracea L. is extracted with hexane:DCM:ethyl-acetate in proportions of 1:1:1 and has a Rf value selected from the group consisting of 0.17, 0.30, 0.36, 0.41, 0.68 and 0.91 when subjected to thin-layer chromatographic fractionation on Silica Gel 60 F254 on aluminum using a solvent of dichloromethane:hexane:methanol in proportions of 1:1:0.2.

181. A method of identifying agents for modulating glucose levels in the blood, the method comprising:
(a) fractionating a Portulaca oleracea L. extract to thereby obtain a plurality of fractions; and
(b) identifying from said plurality of fractions at least one fraction capable of modulating glucose levels in the blood, thereby identifying the agents for modulating glucose levels.

182. The method of claim 181, wherein said fractionating is effected by employing a solvent gradient of increasing polarity

183. The method of claim 182, wherein step (b) is effected by testing an effect of said fraction on:
(i) glucose adsorption through the intestines; and/or
(ii) glucose transport into a cell.
